(19) United States
(12) Patent Application Publication
(10) Pub. No.: US 2014/0294724 A1
(43) Pub. Date: Oct. 2, 2014

(54) COMPOSITIONS AND METHODS FOR TREATMENT OF PROTEINOPATHIES

(71) Applicant: Intellect Neurosciences, Inc., Englewood Cliffs, NJ (US)

(72) Inventor: Daniel G. Chain, New York, NY (US)

(73) Assignee: INTELLECT NEUROSCIENCES, INC., Englewood Cliffs, NJ (US)

(21) Appl. No.: 14/353,650

(22) PCT Filed: Oct. 24, 2012
(86) PCT No.: PCT/US12/61650

§ 371 Date: Apr. 23, 2014

Related U.S. Application Data
(60) Provisional application No. 61/550,625, filed on Oct. 24, 2011.

(51) Int. Cl. A61K 47/48; A61K 49/00

(52) U.S. Cl. CPC A61K 47/48538; A61K 49/0004
USPC 424/1.49; 530/391.7; 530/391.9; 530/391.3; 424/178.1; 424/9.1; 424/181.1; 424/9.6

(57) ABSTRACT

This invention relates to antibody drug conjugates and methods of use thereof. More particularly, antibody drug conjugates comprising a cytoprotective agent are provided, wherein the conjugates are useful for the treatment of proteinopathies such as Alzheimer's disease.

### TECHNICAL FIELD

This invention relates to antibody drug conjugates and methods of use thereof.

### BACKGROUND OF THE INVENTION

Neurodegenerative diseases such as Alzheimer's disease (AD), Parkinson's disease (PD), Huntington's disease (HS), amyotrophic lateral sclerosis (ALS), prion disease, inclusion body myositis and various forms of retinal degeneration such as age related macular degeneration (AMD) have common cellular and molecular mechanisms including protein aggregation, inclusion body formation and oxidative stress leading to inflammation, irreversible tissue damage and ultimately death of nerve cells. The aggregates in these proteinopathies typically consist of fibers containing misfolded protein with a beta-sheet conformation, termed amyloid. Examples of proteins that become misfolded resulting in proteinopathies are beta amyloid, tau, alpha synuclein, prion proteins, superoxide dismutase (SOD), Huntingtin and serum amyloid A. Amyloid or amyloid-like protein aggregates are highly resistant to degradation. β-Amyloid deposits, once formed, are stable even in the absence of ongoing amyloid production. Significantly, amyloid or amyloid-like protein aggregates catalyze the structural conversion of the normally folded protein into additional aggregates via a seeded nucleation-dependent process. Following nucleation, the ongoing production of a ‘normal’ precursor drives additional amyloid formation.

In Alzheimer's disease, Aβ has a partner in tau protein. Tau polypeptides stabilize microtubules. They are abundant in neurons of the central nervous system and are less common elsewhere, but are also expressed at very low levels in CNS astrocytes and oligodendrocytes. When tau proteins are defective, and no longer stabilize microtubules properly, they can cause and/or contribute to proteinopathies (or tauopathies) such as AD and frontotemporal dementias. As tau aggregates accumulate, the dendrite is further sensitized to Aβ induced toxicity—essentially creating a feedback loop whereby tau and amyloid beta increasing push one another to become even more active. This leads to greater aggregation of tau and amyloid beta and the eventual loss of synaptic function and subsequent neuronal death. In non-AD dementia, mutations in tau protein can also have profound effects causing dementia, for example prefrontal dementia.

A novel therapeutic strategy currently under study for AD, AMD and other proteinopathies is the use of antibody based therapies including active vaccines and passive immunotherapy using monoclonal antibodies based on promising preclinical work in various disease models including evidence that IgG antibodies can traverse the blood brain barrier and bind amyloid plaque. Several mechanisms are postulated to account for the decrease in brain amyloid as a result of immunotherapy, including antibody sequestration of Aβ in cerebrospinal fluid and in plaques or in the peripheral circulation, disaggregation of oligomers and plaques and other mechanisms that promote the clearance of Aβ away from the brain. The strategy is most developed in AD where several human clinical trials are being conducted and data being generated. However, although results from preclinical work using amyloid beta immunotherapy were promising, results from human clinical trials have shown that while antibodies engage the target and reduce neurodegeneration as determined by use of biomarkers, such drugs have failed to produce clinical benefit in patients and in certain cases caused brain swelling or inflammation. These data indicate the need for both earlier intervention and improved therapies.

Monotherapy may not be effective to treat complex diseases such as AD and other proteinopathies. It is likely that the successful treatment of such diseases will require the concomitant application of neuroprotective agents. Thus, a number of factors may limit the effectiveness of amyloid lowering treatments if applied in isolation. First, the degree to which amyloid beta levels need to be reduced to delay onset or slow progression is unknown. If amyloid beta concentrations are several-fold above those capable of causing neuronal degeneration, a large proportionate reduction in levels might be insufficient to slow degeneration. Second, the ideal scenario would include the application of amyloid beta lowering immunotherapy drugs in early stages of amyloid beta accumulation, i.e. years before onset of symptoms. This approach would require drugs of exceptionally low toxicity administered with difficult to achieve high compliance rates years before clinical manifestations begin. Third, amyloid-based therapies are unlikely to improve function or plasticity of damaged but surviving neurons. Fourth, amyloid associated proteins such as apolipoprotein E4 can increase the pathogenicity of the amyloidogenic protein either by increasing rate of fibrillogenesis or by other mechanism. Finally, although the bulk of current evidence points to amyloid beta accumulation as a critical primary causative factor in AD, a number of other mechanisms might constitute important causative factors as well. Such non amyloid beta mechanisms, such as abnormal tau protein, might play synergistic roles as the disease progresses. Thus, parallel neuroprotective strategies can play a vital role in delaying AD and other proteinopathies and slowing disease progression.

Combination therapy in which different drugs are administered simultaneously is a formidable challenge to the pharmaceutical industry from a regulatory standpoint in addition to pharmacological and other considerations. Thus, before investigational new drugs can be combined into a single therapy, each drug needs to be tested for safety alone before it is tested in combination involving a costly clinical trial process. Drugs may have markedly different bioavailability and pharmacokinetics such that dosing regimens for combination drugs can be cumbersome and even incompatible. Another problem, with combination therapies is the need for two different formulations adding to the cost and complexity of development ensuring that the formulations are compatible. The interpretation of data from clinical trials involving combination therapies with drugs that interact independently of each other can be difficult. It would be desirable to overcome these problems associated with combination therapies.

Despite advances in understanding mechanisms driving neurodegenerative, amyloidogenic diseases, such as AD, PD and other proteinopathies, there is a need for additional and improved treatments. There is a need, in particular, for improved therapeutic reagents and methods for the treatment of proteinopathies, and, in particular, reagents that can provide cytoprotective (e.g., neuroprotective) benefits to prevent protein aggregation, clear amyloid and counter the detrimental effects of oxidative stress, caused e.g., by oxidotoxins, as well as inhibit Aβ and/or tau-induced neuronal toxicity.

### SUMMARY OF THE INVENTION

An object of the invention is to provide improved therapeutic reagents and methods for the treatment of proteinopathies.

In certain aspects, an object of the invention is to provide antibody-drug conjugates (ADC) with neuroprotective properties to reduce inflammation and oxidative stress in the brain, including in cerebrospinal fluid (CSF) and blood vasculature, in the eye, and in peripheral tissues such as the kidney and liver.

An object of the invention is to provide amyloid clearing antibodies with neuroprotective properties to reduce inflammation. Another object of the invention is to enhance the clearing capacity of an antibody by combining the antibody with a small molecule inhibitor of protein aggregation.

An object of the invention is to provide a high affinity conjugated antibody (KDa >10⁻⁹ M) that binds pre-fibrillar amyloid and does not significantly bind amyloid precursor proteins.

An object of the invention is to provide a high affinity conjugated antibody (KDa >10⁻⁹M) that binds fibrillar amyloid and does not provoke Fc mediated phagocytosis or complement activation.

An object of the invention is to provide an antibody specific to a neoepitope in a target protein, e.g., a free amino or carboxyl group created by cleavage of a peptide bond in the precursor protein.

An object of the invention is to reduce or prevent side effects due to dissolution of plaques following treatment with immunotherapy or other amyloid decreasing drugs, that can lead to vascular deposition of newly released Aβ fragments, which gives rise to inflammation and vasogenic edema.

An object of the invention is to provide antioxidant on-site delivery to counteract the foci of inflammation leading to vasogenic edema. Another object of the invention is to provide cytoprotective agents (e.g., antioxidants) to sites of inflammation around pre-existing plaque or resulting from soluble Aβ species.

An object of the invention is to prevent or reduce binding of the cytoprotective agent (e.g., antioxidant) to endogenous receptors by conjugating the cytoprotective agent to the antibody through the receptor binding site, e.g. for melatonin, conjugation of N-acetyl-5-methoxytryptamine through C1 of the indole ring thereby preventing binding to the melatonin receptor.

An object of the invention is to provide compositions comprising an antibody-drug conjugate (ADC), said ADC comprising an antibody targeted to an amyloidogenic polypeptide or a tau polypeptide conjugated to a cytoprotective agent. In certain embodiments, the amyloidogenic polypeptide is an amyloid beta peptide. In certain embodiments, the amyloidogenic polypeptide is an amyloid-associated polypeptide selected from the group consisting of protease inhibitor alpha 1-antichymotrypsin, apolipoprotein E (apoE), and EpoE4. The tau polypeptide may be hyperphosphorylated. In yet other embodiments, the amyloidogenic polypeptide is selected from the group consisting of prion (PrPSc), amylin, calcitonin, atrial natriuretic factor (AANF), apolipoprotein AI, serum amyloid A, medin, transthyretin, lysozyme, beta 2 microglobulin (Aβ2M), gelsolin, keratoepithelin, cystatin, immunoglobulin light chain AL, α-synuclein, Huntingtin, and superoxide dismutase.

In certain embodiments, an antibody in an ADC may be humanized. In certain embodiments, the antibody is a monoclonal antibody, a humanized antibody, a chimeric antibody, a bispecific antibody, an artificial antibody, a scFv antibody or a F(ab), or fragment thereof. In yet other embodiments, the antibody may be a camel antibody.

In an embodiment, the cytoprotective agent in an ADC of the invention is an antioxidant. An example of an antioxidant is melatonin or analog thereof. In some embodiments, the antioxidant is selected from the group of antioxidants that possess one of more of the following properties: (1) capable of neutralizing free radicals e.g. hydroxyl radicals; (2) capable of preventing generation of ROS through metal chelation; (3) capable of avoiding the formation of pro-oxidant intermediates; (4) capable of increasing mitochondrial metabolism: (5) capable of increasing glucose utilization. Melatonin and indole-3-propionic acid are examples of preferable antioxidants based on their multifunctional antioxidant activities. Additional antioxidants include but are not limited to the following group of compounds and derivatives: an indole amine, an indole acid, vitamin E, vitamin C, lipoic acid, uric acid, curcumin, glutathione, a polyphenol, a flavonoid, an anthraquinone methylthioninium chloride, dimebone, a rhodamine, an insulin sensitizer e.g. pioglitazone and rosiglitazone, an 8-hydroxyquinolone derivative e.g. PBT2, PBT434, penicillamine, Trientine, a tetracycline, (N-(pyridin-2-ylmethyl)aniline), and N1,N1-dimethyl-N4-(pyridin-2-ylmethyl)benzene-1,4-diamine, 2,6-diaminopyridine. In certain embodiments, the antioxidant is selected from the group consisting of idebenone, cyclohexane-1,2,3,4,5,6-hexyl, myo-inositol, Scyllo-inositol, and an NO scavenger.

In certain embodiments the cytoprotective agent is an inhibitor of protein aggregation acting through metal binding or intercalating with prefibrillar protein. Melatonin is an example of a preferable protein aggregation inhibitor. Thus, melatonin added to beta amyloid in the presence of apoE4 results in a potent isoform-specific inhibitor of fibril formation, the extent of which is far greater than that of the inhibition produced by melatonin alone. Additional antioxidants include but are not limited to the following group of compounds and derivatives: AZD-103, cyclohexane-1,2,3,4,5,6-hexyl, myo-inositol, scyllo-inositol, indole-3-propionic acid, an indole amine, an indole acid, an 8-hydroxyquinolone derivative e.g. PBT2, PBT434, penicillamine, Trientine, a tetracycline, (N-(pyridin-2-ylmethyl)aniline), N1,N1-dimethyl-N4-(pyridin-2-ylmethyl)benzene-1,4-diamine, 2,6-diaminopyridine, methylene blue, and TRx0014.

In certain embodiments the cytoprotective agent lowers production of amyloid. Useful amyloid lowering compounds include but are not limited to a gama secretase inhibitor or modulating factor, a beta secretase inhibitor or modulating factor, ponatinib.

In certain embodiments the cytoprotective agent is proneurogenic. Such proneurogenic compounds include but are not limited to P7C3A20 and P763.

In certain embodiments, an antibody in an ADC of the invention is conjugated to a cytoprotective agent by a linker. Preferably, the linker is selected from the group consisting of hydrazone linker, disulfide linker, thioether linker, and peptide linker. In one embodiment, the linker is cleavable under intracellular conditions. The cleavable linker may be a peptide linker cleavable by an intracellular protease. A peptide linker can be a dipeptide linker. The peptide linker can be a citrulline-valine based linker.

In certain embodiments, the ADC of the invention comprises a marker. The marker can be selected from the group consisting of an isotope, a radiolabel, a fluorescent label, and an enzyme that catalyzes a detectable modification to a substrate. The marker can be conjugated to a cytoprotective agent comprised in the ADC. The marker may be incorporated into the cytoprotective agent during synthesis of the cytoprotective agent.

In other embodiments, a pharmaceutical formulation comprising an ADC as described above, and a pharmaceutically acceptable carrier is provided. In a specific embodiment, a pharmaceutical composition comprises an ADC comprising an antibody targeted to an amyloidogenic polypeptide or a tau polypeptide conjugated to a cytoprotective agent, and a pharmaceutically acceptable carrier.

In certain embodiments, a method for detecting an amyloid deposit in a subject is provided, the method comprising administering a composition comprising an ADC comprising an antibody targeted to an amyloidogenic polypeptide or a tau polypeptide conjugated to a cytoprotective agent and a marker, and detecting the presence of the marker, wherein the subject has an amyloid deposit if the marker is detected in the subject.

In other embodiments, a method for inhibiting accumulation of an amyloidogenic polypeptide in the brain of a patient suffering from a proteinopathy is provided, the method comprising administering to a patient in need thereof a therapeutically effective amount of a composition comprising an ADC, wherein the ADC comprises an antibody targeted to an amyloidogenic polypeptide or a tau polypeptide conjugated to a cytoprotective agent.

In another embodiment, a method for promoting clearance of aggregates from the brain of a subject is provided, the method comprising administering to the subject an ADC-containing composition provided above, such as, e.g., a composition comprising an ADC comprising an antibody targeted to a tau polypeptide, under conditions and in an amount effective to promote clearance of neurofibrillary tangles from the brain of the subject.

In yet another embodiment, a method for treating or delaying onset of a proteinopathy is provided, the method comprising administering to a subject in need thereof an effective amount of an ADC-containing composition provided above, e.g., a composition comprising an ADC comprising an antibody targeted to an amyloidogenic polypeptide or a tau polypeptide conjugated to a cytoprotective agent, for inhibiting the formation of fibrils, the formation of amyloid or amyloid-like deposits, or to inhibit the formation of neurofibrillary tangles.

A proteinopathy for treatment according to any of the methods provided above may be selected from the group consisting of age related macular degeneration (AMD), glaucoma, traumatic brain injury, cerebral amyloid angiopathy (CAA), hereditary cerebral hemorrhage with amyloidosis Dutch type (HCHWA-D) Alzheimer's disease, early onset familial Alzheimer's disease (EOFAD), Down Syndrome, Parkinson's disease (PD), Huntington's disease (HD), amyotrophic lateral sclerosis (ALS), transmissible spongiform encephalopathy, Pick's complex, prion disease, peripheral tissue amyloidosis (e.g., liver and kidney), and serum amyloidosis.

In any of the above embodiments, a subject administered an ADC-containing composition of the invention may be a mammal. In a specific embodiment, the mammal is a human. In certain embodiments, an ADC-containing composition of the invention, as described above is administered intravenously, subcutaneously, intradermally, intramuscularly, intaperitoneally, intracerebrally, intranasally, orally, transdermally, buccally, intra-arterially, intracranially, or intracephalically.

The details of one or more embodiments of the invention are set forth in the description below. Other features, objects, and advantages of the invention will be apparent from the description and from the claims.

### BRIEF DESCRIPTION OF THE DRAWINGS

FIG. 1 shows antibody-drug conjugates of the invention. In particular, FIG. 1A depicts an antibody conjugated to melatonin using maleimide-based conjugation through cysteine side chains on the antibody. FIG. 1B depicts an antibody conjugated to melatonin via conjugation though lysine side-chains on the antibody via a Mannich reaction. FIG. 1C depicts an antibody conjugated to a melatonin-PEG derivative using maleimide-based conjugation through cysteine side-chains on the antibody. FIG. 1D depicts an antibody conjugated to melatonin via maleimide-based conjugation through lysine side chains on the antibody modified with 2-iminothiolane.

FIG. 2 shows the results of experiments testing the capacity of constructs of the invention to reduce β-amyloid fibrillogenisis as evaluated by Thioflavin T Spectrofluorometry. In particular, FIG. 2A shows florescence intensities of tested samples measured at an excitation wavelength of 450 nm and an emission wavelength of 485 nm at t=0. FIG. 2B shows florescence data at t=0 normalized as a percentage of a control sample. FIG. 2C shows florescence intensities of tested samples measured at an excitation wavelength of 450 nm and an emission wavelength of 485 nm at t=24 hours. FIG. 2D shows a time scan of fluorescence intensity at t=24 hours normalized as a percentage of a control sample.

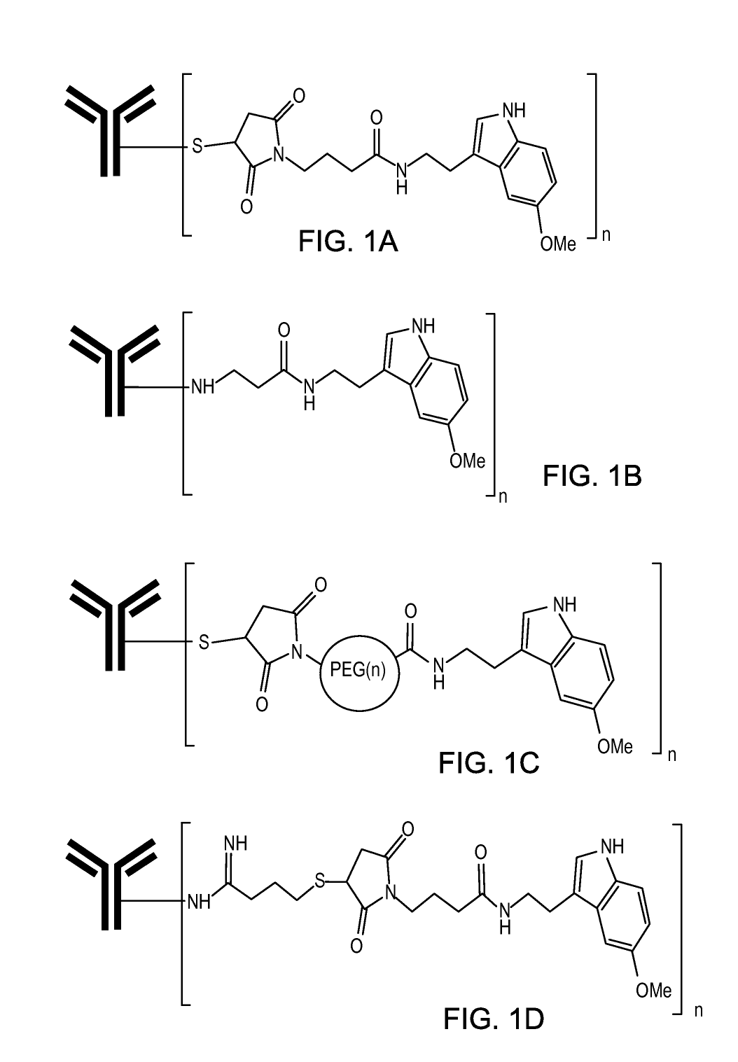

### DETAILED DESCRIPTION

### I. Overview

Improved compositions and methods are needed for the treatment of neurodegenerative diseases such as, but not limited to, Alzheimer's disease (AD), Parkinson's disease (PD), Huntington's disease (HD), amyotrophic lateral sclerosis (ALS) and prion diseases, diseases which are referred to herein generally as proteinopathies. Certain types of neuroprotective molecules that have antioxidant or anti-aggregation properties can block beta amyloid neurotoxicity by a variety of mechanisms including metal-chelation, free radical scavenging activity, mitochondrial activation, insulin sensitization and other mechanisms. For example the antioxidant melatonin can interact with the high affinity copper binding site in Aβ and inhibit fibril formation, and can provide cytoprotective effects to neuronal cells in AD and other proteinopathies. See, U.S. Pat. No. 5,958,964.

The invention is based in part on the discovery that antibody-drug conjugates (termed “ADC” herein) of an antioxidant molecule (or molecule that inhibits aggregation) and antibody directed to an antibody targeted to a specific polypeptide involved in the etiology of a proteinopathy are improved therapeutics because the amyloid clearing antibody is thereby equipped with additional neuroprotective properties to reduce oxidative damage and in some cases also inhibit aggregation or promote disaggregation. As one example, and without limitation, melatonin, when conjugated to an antibody targeted to a specific polypeptide involved in the etiology of a proteinopathy (e.g., amyloid β peptide or Tau), can provide an effective therapeutic reagent. Without being bound by theory, ADC can recognize and remove toxic amyloidogenic polypeptides and polypeptides that cause neurofibrillary tangles (e.g., tau) while also providing cytoprotective benefits to the target cells (e.g., dendrites or other neuronal cells). Thus, in certain embodiments, a composition comprising an antibody which is targeted to an amyloid polypeptide (e.g., amyloid β peptide) or to tau or another misfolded protein, wherein said antibody is conjugated to a cytoprotective agent (e.g., an antioxidant such as melatonin), is provided.

In a specific embodiment, an ADC of the invention comprises an Aβ targeted antibody conjugated to melatonin. In another embodiment, an ADC of the invention comprises a tau polypeptide-targeted antibody conjugated to melatonin. In some instances antibody in the ADC is preferably conjugated to melatonin via a linker, such as, but not limited to a hydrazone linker, disulfide linker, thioether linker, or peptide linker. Alternatively, antibody in the ADC is preferably conjugated to melatonin via solvent exposed lysine residues using maleimide based linkers (see experimental data) or using a Mannich type reaction.

Also provided are methods for inhibiting accumulation of amyloid β peptide or hyperphosphorylated or cleaved tau polypeptide in the brain of a patient suffering from a proteinopathy, comprising contacting amyloid β peptide or hyperphosphorylated tau polypeptide in the brain of the patient with an ADC composition provided herein.

In yet another embodiment, a method for treating or delaying onset of a proteinopathy (e.g., age related macular degeneration (AMD), glaucoma, traumatic brain injury, cerebral amyloid angiopathy (CAA), hereditary cerebral hemorrhage with amyloidosis Dutch type (HCHWA-D) AD, early onset familial AD (EOFAD), Down Syndrome, Parkinson's disease (PD), Huntington's disease (HD), amyotrophic lateral sclerosis (ALS), prion disease, etc.), comprising administering to a subject in need thereof an effective amount of an ADC composition according to the present invention, to inhibit the formation of fibrils or the formation of amyloid or amyloid-like deposits associated with amyloidosis-related diseases, or to inhibit the formation of neurofibrillary tangles associated with tauopathies. Non-neuropathic amyloidogenic diseases and disorders may also be treated according to the methods provided herein, as described in detail, infra.

Alzheimer's disease (AD) is a dementing illness with progressive loss of memory, task performance, speech and recognition of people and objects. There is extensive degeneration of neurons especially in the basal forebrain and hippocampus. At least as important for pathogenesis may be the synaptic pathology and altered neuronal connections. Pathological manifestations of AD include extracellular plaques of β-amyloid and intracellular neurofibrillary tangles composed of abnormally bundled cytoskeletal fibers. AD involves two major kinds of protein aggregates. Extracellular aggregates known as neuritic plaques are comprised primarily of the beta amyloid peptide, which is derived by the proteolytic processing of the amyloid precursor protein, APP. The beta amyloid peptides have beta sheet structures. There are also intracellular aggregates of the microtubule associated protein—tau. The deposition of amyloid plaques is thought to destabilize neurons by mechanisms which require further clarification. Tangles are associated with hyperphosphorylation of tau, a microtubule-associated protein, and of neurofilament H/M subunits, processes that lead to misfolding and accumulation of these proteins, along with a disruption of microtubules.

Deposition of cerebral amyloid is a primary neuropathologic marker of AD. Thisamyloid is composed of a 40-42 amino acid peptide called the amyloid beta protein (Aβ). Amyloid deposits in AD are found mainly as components of senile plaques, and in the walls of cerebral and meningeal blood vessels, where they gradually become dystrophic and cause damage through inflammation and oxidative stress. In addition, various soluble toxic Aβ species, including oligomers and fibrils, are found in cerebrospinal fluid. As the concentration of fibrils increases, they eventually deposit as plaque on the surface of cells.

The discovery that AD could be inherited in an autosomal dominant fashion due to a mutation in the gene encoding APP was a seminal event in AD research. Amyloid-β peptide is excised from APP via sequential scission by the β-APP cleaving enzyme (BACE) and γ-secretase. These observations led to the articulation of the amyloid cascade hypothesis. This hypothesis was further supported by the discovery that AD could also be caused by autosomal dominant mutations in presenilin 1 (PSEN1) and PSEN2, which are both homologous proteins that can form the catalytic active site of gamma-secretase.

In Alzheimer's disease, Aβ has a partner in tau protein. Tau polypeptides stabilize microtubules. They are abundant in neurons of the central nervous system and are less common elsewhere, but are also expressed at very low levels in CNS astrocytes and oligodendrocytes. When tau proteins are defective, and no longer stabilize microtubules properly, they can cause and/or contribute to proteinopathies (or tauopathies) such as AD and frontotemporal dementias. In the early phase, neurofibrillary tangles accumulate around the dendrite and begin to damage the dendrite. Although there is much evidence linking tau to neurodegeneration, the precise mechanism of tau-mediated neurotoxicity remains to be elucidated. For many years, it was assumed that NFTs were the cause of neuronal toxicity. However, in some animal models overexpressing tau, neurodegeneration has been demonstrated in the absence of overt NFT pathology. Additionally, recent evidence suggests that memory function and neuronal loss can be restored in a tauopathy mouse model despite the ongoing accumulation of NFTs. Moreover, NFTs have been suggested to persist in neurons for 20-30 years making them unlikely candidates for catalysing immediate toxicity. In fact, a large immunohistochemical study on cholinergic basal forebrain neurons in the nucleus basal is using an early tau marker demonstrated pre-tangle neurons, and NT staining correlates extremely well with cognitive decline, which occurs before the emergence of significant NFT pathology. Finally, synaptic loss correlates better with cognitive decline than NFTs, again suggesting the possibility of different mechanisms for tau toxicity: At least two forms of pathologic tau preceded tangle formation, oligomers which occur at the very earliest stages of the disease and a truncated form “delta tau” which is produced by caspase cleavage on the intact protein.

As tau aggregates accumulate, the dendrite is further sensitized to Aβ induced toxicity—essentially creating a feedback loop whereby tau and amyloid beta increasing push one another to become even more active. This leads to greater aggregation of tau and amyloid beta and the eventual loss of synaptic function and subsequent neuronal death. In non-AD dementia, mutations in tau protein can also have profound effects causing dementia, for example prefrontal dementia.

A number of neurological diseases are known to have filamentous cellular inclusions containing microtubule associated protein tau, e.g., Alzheimer's disease (AD), progressive supranuclear palsy (PSP), corticobasal degeneration (CBD), Pick's disease (PiD) and a group of related disorders collectively termed frontotemporal dementia with Parkinsonism linked to chromosome 17 (FTDP-17), amyotropic lateral sclerosis (ALS), Creutzfeldt-Jakob disease (CJD), dementia pugilistica (DP), GerstmannStraussler-Scheinker disease (GSSD), Lewy body disease and Huntington disease. Although the etiology, clinical symptoms, pathologic findings and the biochemical composition of inclusions in these diseases are different, there is emerging evidence suggesting that the mechanisms involved in aggregation of normal cellular proteins to form various filamentous inclusions are comparable. It is believed, that an initial alteration in conformation of microtubule associated protein tau, which initiates generation of nuclei or seeds for filament assembly, is one of the key features. This process can be influenced by the posttranslational modification of normal proteins, by mutation or deletion of certain genes and by factors that bind normal proteins and thus alter their conformation.

Inheritance of apoE4 is a strong risk factor for the development of late-onset sporadic Alzheimer's disease. Several lines of evidence suggest that apoE4 binds to the Alzheimer Abeta protein and, under certain experimental conditions, promotes formation of beta-sheet structures and amyloid fibrils.

AD pathology has many similarities to changes that are responsible for retinal degeneration including glaucoma, diabetic retinopathy, Bests disease and both dry and wet forms of age related macular degeneration. An important characteristic common to between AD and AMD for example, is the presence of amyloid β in the senile plaques of the AD brain and in the drusen of AMD patients. Aβ is a key regulator of the progression from drusen to AMD causing an imbalance of angiogenesis-related factors in the retinal pigment epithelial (RPE) cells. Mice that lack the Aβ-degrading enzyme neprilysin develop RPE degeneration, and the sub-RPE deposits that are formed have features similar to those of AMD in humans. Moreover, changes in the concentrations of Aβ and Tau in the vitreal fluid of the eye mirror changes in the cerebral spinal fluid (CSF) of Alzheimer's patients where Aβ reduction is accompanied by a concomitant increase in tau protein. These data suggest that a common pathogenic mechanism exist between AMD and AD. Thus, therapeutic approaches that have targeted Aβ in patients with AD can also be applied to AMD and possibly other forms of retinal degeneration.

Parkinson's disease (PD) is characterized by resting tremor, rigidity, slow movements and other features such as postural and autonomic instability. It is caused by degeneration of dopaminergic neurons in the substantia nigra of the midbrain and other monoaminergic neurons in the brain stem. The discovery of several genes in which mutations cause early onset forms of PD has greatly accelerated research and led to a better understanding of the disease. Point mutations or increased gene dosage of the alpha synuclein gene cause autosomal dominant PD by a gain of function mechanism. Recessive early onset PD can be caused by point mutations in the genes encoding parkin, DJ1 or PINK1, presumably by loss of function mechanisms. The pathological hallmark feature of adult PD is the Lewy Body, an inclusion found in cytoplasm of neurons. Lewy Bodies are densest in the substantia nigra but can also be present in monoaminergic, cerebral cortical and other neurons. There are also aggregates in neuritis, which are referred to as Lewy neurites. A major constituent of Lewy bodies is aggregated alpha synuclein protein.

Huntington disease (HD) is a progressive neurodegenerative disorder caused by expansion of CAG repeat coding for polyglutamine in the N-terminus of the huntingtin protein. There is a striking threshold between the threshold for aggregation in vitro and threshold for disease in humans. Inclusions containing Huntingtin are present in regions of the brain that are most degenerate. Huntingtin aggregates can be labeled with antibodies to the N-terminus of the protein, or antibodies to ubiquitin, a marker of misfolded proteins and a signal for degradation by the proteasome. Proteasomes may have difficulty digesting them, however, leading to the accumulation of aggregates. The aggregates contain fibers and appear to have a beta sheet characteristic of beta amyloid. Other proteins such as Creb binding protein may be recruited into Huntingtin aggregates.

Amyotrophic lateral sclerosis (ALS) is a progressive fatal disease caused by degeneration of lower motor neurons in the horn of the spinal cord and upper motor neurons of the cerebral cortex, resulting in progressive motor weakness. Rare early onset familial forms of the disorder can be caused by mutations in the superoxide dismutase gene which cause aggregation of the protein in inclusions.

Neurodegenerative diseases caused by prions can be sporadic or can be acquired by either environmental transmission or via genetic mutations. Pathology can include amyloid plaques which appear similar to those of AD except that they are formed by prion proteins. Prion aggregation can take place both extracellularly and intracellularly.

The important role of oxidative stress, an imbalance between the production and detoxification of oxidative reaction products continues to be the subject of extensive research in AD. While oxidation products accumulate to some degree in normal brain, their levels increase with age and are substantially higher in AD, including in its early stages. Excessive levels of hydrogen peroxide and ROS such as hydroxyl free radical and superoxide lead to the formation of oxidation products including oxidized proteins, lipid peroxides, advanced glycosylation end products and DNA adducts. Protein and lipid oxidation leads to loss of critical enzyme functions, including those regulating glutamate transport, which results in excitotoxicity due to excessive extracellular glutamate, and to the loss of ATPases, causing disruption of calcium homeostasis and impaired mitochondrial function. Oxidative stress triggers degenerative signaling, including activation of stress kinases and caspases. Sources of oxidative stress in AD include impaired mitochondrial metabolism, decreases glucose utilization and A13. The peptide may be a major source of ROS when binding Cu2+ or Fe3+ and also by interacting with RAGE receptor at the cell surface to promote lipid peroxidation.

With regard to oxidative stress, the pro-oxidant properties of the free amyloid-β molecule (Aβ) may lead to hydroxyl radical-induced cell death (e.g., by Fenton and Fenton-like reactions). Additionally, Aβ initiates flavoenzyme-dependent rises in intracellular H₂O₂ and lipid peroxides, which also cause radical generation. Rises in Aβ protein have been shown to induce oxidative stress. Impairment of neurotrophin activity on associated tyrosine kinase receptors has been suggested to represent an important factor in AD pathology. Moreover, the pathophysiological phenomena associated with AD have also been found to be associated with other neurodegenerative disorders such as Parkinson's disease (PD), Huntington's disease (HD), amyotrophic lateral sclerosis (ALS), prion diseases, Pick's complex, and other proteinopathies. See, Srinivasan et al. (2006) Behavioral and Brain Functions, 2:15; Ross and Poirier (2004) Nat Med Rev; 510-517.

### II. Definitions

As used herein, the term “amyloidogenic polypeptide” means a polypeptide that is capable of forming amyloid fibrils, filaments, and/or amyloid deposits, such as, but not limited to, amyloid β peptide (Aβ), amylin, calcitonin, atrial natriuretic factor, apolipoprotein AI, serum amyloid A, etc. Amyloid fibrils are insoluble proteinaceous fibrillar aggregates with a characteristic structure (the cross-β core) that form and deposit in more than 40 pathological conditions in humans. As used herein, “amyloid beta peptide” (Aβ) refers to the 40-42 amino acid peptide that makes up the cerebral amyloid which is the primary neuropathologic marker of AD and other amyloidogenic diseases, and includes fragments of Aβ capable of causing cytotoxic effects on cells. For example, one such fragment of Aβ is the fragment made of up amino acid residues 25-35 of Aβ. See, Glenner, G. G., and Wong, C. W. (1984) Biochem Biophys Res Commun 120:885-890, for the full amino acid sequence of Aβ.

As used herein, “amyloid-associated polypeptide” includes polypeptides that may be found in amyloid deposits, such as, but not limited to, the protease inhibitor alpha 1-antichymotrypsin and the lipid transport protein apolipoprotein E (apoE), which are intimately associated with Aβ, e.g., in the filamentous amyloid deposits of AD, and also includes amyloid-promoting factors (pathological chaperones) such as, but not limited to, EpoE4.

As used herein, the term “antibody” means an immunoglobulin protein, which is capable of binding an antigen, and includes full length antibodies, monoclonal antibodies (including full length monoclonal antibodies), polyclonal antibodies, multi-specific antibodies (e.g., bispecific antibodies), human, humanized or chimeric antibodies, unibody and antibody fragments, e.g., Fab fragments, F(ab′), Fab′, F(ab′)2, and Fv fragment, fragments produced by a Fab expression library, epitope-binding fragments of any of the above, and engineered forms of antibodies, e.g., scFv molecules, so long as they exhibit the desired activity, e.g., binding to the desired target. The term also includes single chain antibodies in which heavy and light chain variable domains are linked through a spacer. Thus, for example, non-limiting examples of an antibody according to the present invention include an antibody which is targeted to (i.e., capable of binding to) an amyloid polypeptide (e.g., amyloid β peptide), to an amyloid-associated polypeptide, or to tau. An antibody that is “targeted to” a polypeptide, as used herein, is an antibody that specifically recognizes and binds to that polypeptide.

The term “humanized antibody” means an antibody in which the complementary-determining regions (CDRs) of a mouse or other non-human antibody are grafted onto a human antibody framework. By human antibody framework is meant the entire human antibody excluding the CDRs.

The term “chimeric antibody” refers to an antibody in which the whole of the variable regions of a mouse or rat antibody are expressed along with human constant regions.

The term “free end specific” means a molecule which preferentially binds to a particular free terminus/end of a peptide or protein. For example, in the context of an amyloid β peptide, the term “free end specific” refers to binding specifically to a free terminus of an amyloid β peptide or to any fragment thereof to slow down or prevent the accumulation of amyloid β peptides in the extracellular space, interstitial fluid and/or cerebrospinal fluid and to block the interaction of Aβ peptides with other molecules that contribute to the neurotoxicity of Aβ. The term “free end specific” can be used in reference to any amyloidogenic protein fragment, including, for example, the C-terminal portion of delta Tau, Huntingtin cleavage products, Abeta protein, and the like.

As used herein, the term “cytoprotective agent” refers to an agent that is capable of ameliorating one or more deleterious effects in a cell, such as, e.g., a neuronal cell, that are caused by oxidotoxins, oxidative stress (e.g., caused by amyloid (3 and/or mitochondrial dysfunction), and/or one or more other processes associated with proteinopathies (i.e., diseases or disorders associated with protein misfolding and/or amyloid deposition and/or neurofibrillary tangles). By way of example, and without limitation, a cytoprotective agent can be an antioxidant, such as, but not limited to, melatonin, an indole acid, an indole amine, an indole acid, vitamin E, vitamin C, lipoic acid, uric acid, curcumin, glutathione, a polyphenol, a flavonoid, an anthraquinone methylthioninium chloride, dimebone, a rhodamine, an insulin sensitizer e.g. pioglitazone and rosiglitazone, an 8-hydroxyquinolone derivative e.g. PBT2, PBT434, penicillamine, Trientine, a tetracycline, (N-(pyridin-2-ylmethyl)aniline), N1,N1-dimethyl-N4-(pyridin-2-ylmethyl)benzene-1,4-diamine, 2,6-diaminopyridine, idebenone, cyclohexane-1,2,3,4,5,6-hexyl, myo-inositol, Scyllo-inositol, NO scavengers, and other antifibrillogenic molecules.

As further used herein, “melatonin” refers to the compound N-[2-(5-Methoxyindol-3-yl)ethyl]acetamide (also referred to as N-acetyl-5-methoxytryptamine). Melatonin analogs that retain the function of preventing the cytotoxic effects of Aβ are well known in the art. Melatonin and such analogs are described in greater detail, infra.

As used herein, the term “hydrazine linker” refers to a linker moiety that, upon a change in condition, such as a shift in pH, will undergo a cyclization reaction and form one or more rings. The hydrazine moiety is converted to a hydrazone when attached. This attachment can occur, for example, through a reaction with a ketone group on the L moiety. Therefore, the term “hydrazone linker” can also be used to describe the hydrazine linkers of the invention.

Within the meaning of the present invention, the term “inhibit” and its grammatical variations are used to refer to any level of reduction in a function or amount. For example, when used in relation to “inhibiting accumulation of amyloid P polypeptides or amyloid-associated polypeptides” in a subject, “inhibit” means any level of reduction of the number of any species of amyloid P polypeptides in the subject and/or any level of reduction of the size and/or frequency of amyloid deposits in the subject.

As used herein, the terms “treat”, “treatment”, and the like mean to relieve or alleviate at least one symptom associated with a condition (e.g., proteinopathy), or to slow or reverse the progression of such condition. For example, in relation to Aβ related diseases such as, but not limited to, AD, PD, or HD, the term “treat” may mean to relieve or alleviate at least one symptom of the disease, such as, for example, and without limitation, memory loss and/or confusion, inability to recognize family and friends, inability to learn new things, difficulty carrying out tasks that involve multiple steps (such as getting dressed), problems coping with new situations, delusions and paranoia, and/or impulsive behavior (for AD); involuntary trembling, rigid or stiff muscles, loss of ability to make rapid, spontaneous movements, gait characterized by bent or flexed body, difficult in maintaining balance (for PD); movement disorders such as involuntary jerking or writhing movements (chorea), involuntary, sustained contracture of muscles (dystonia), muscle rigidity, slow, uncoordinated fine movements, slow or abnormal eye movements, impaired gait, posture and balance, difficulty with the physical production of speech, and/or difficulty swallowing and cognitive disorders, such as difficulty planning, organizing and prioritizing tasks, inability to start a task or conversation, lack of flexibility, or the tendency to get stuck on a thought, behavior or action (perseveration), lack of impulse control that can result in outbursts, acting without thinking and sexual promiscuity, problems with spatial perception that can result in falls, clumsiness or accidents, lack of awareness of one's own behaviors and abilities, difficulty focusing on a task for long periods, slowness in processing thoughts or “finding” words, and/or difficulty in learning new information (for HD). Those of skill in the art will appreciate that the symptoms of the proteinopathies that can be treated according to the present invention are well known in art, and the foregoing are provided by way of non-limiting example. The skilled artisan (e.g., an individual's physician) will understand whether one or more symptoms of a proteinopathy contemplated for treatment herein have been treated upon examination of the individual.

In certain aspects, the compounds and compositions described herein may be used to delay the onset and/or reduce the risk of developing or worsening a disease. For delayed onset of a proteinopathy, as provided herein, preferably, an ADC of the invention will delay the onset of the disease (i.e., appearance of clinical manifestation of the disease, or appearance of increased symptoms of the disease after initial (early) signs of the disease are observed, e.g., by the subject or the subject's physician) by at least 1 year, at least 2 years, at least 3 years, at least 4 years, at least 5 years, at least 6 years, at least 7 years, or longer. Thus, for example, if a subject is suspected to be in early stages of AD or at risk of developing AD at the age of 61, treatment of the subject with an ADC or ADC-containing composition of the invention, preferably, although not necessarily, would delay the onset at least until the age of 62 or older.

As used herein, the term “therapeutically effective” applied to dose or amount refers to that quantity of a compound or composition (e.g., pharmaceutical composition) that is sufficient to result in a desired activity upon administration to an animal in need thereof. Thus, within the context of the present invention, the term “therapeutically effective amount” refers to that quantity of a compound or composition that is sufficient to treat at least one symptom of a proteinopathy, as described above. When a combination of active ingredients is administered, a therapeutically effective amount of the combination may or may not include amounts of each ingredient that would have been effective if administered individually. A therapeutically effective amount does not necessarily occur by administration of one dose, and may occur only after administration of a series of doses. Thus, a therapeutically effective amount may be administered in one or more administrations.

As used herein “combination therapy” or “adjunct therapy” means that the individual in need of the ADC-containing composition is treated or given another drug or therapeutic agent (“2^{nd} composition”) for the disease in conjunction with the ADC-containing composition. Combination therapy can be sequential therapy where the individual (e.g., patient) is treated first with one composition and then the second composition, or the two are given simultaneously. These compositions are said to be “coadministered.”

The phrase “pharmaceutically acceptable” refers to molecular entities and compositions that are physiologically tolerable when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The terms “express” and “expression” mean allowing or causing the information in a gene or DNA sequence to become manifest, for example, producing an non-coding (untranslated) RNA or a protein by activating the cellular functions involved in transcription and translation of a corresponding gene or DNA sequence. A DNA sequence is expressed in or by a cell to form an “expression product” such as RNA or a protein. The expression product itself, e.g. the resulting RNA or protein, may also be said to be “expressed” by the cell.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences. In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990; 87:2264, modified as in Karlin and Altschul, Proc. Natl. Acad. Sci. USA 1993; 90:5873-5877. Such an algorithm is incorporated into the NBLAST and XBLAST programs of Altschul et al., J. Mol. Biol. 1990; 215:403. BLAST nucleotide searches can be performed with the NBLAST program, score=100, word length=12, to obtain nucleotide sequences homologous to sequences of the invention. BLAST protein searches can be performed with the XBLAST program, score=50, word length=3, to obtain amino acid sequences homologous to protein sequences of the invention. To obtain gapped alignments for comparison purposes, Gapped BLAST can be utilized as described in Altschul et al., Nucleic Acids Res. 1997; 25:3389. Alternatively, PSI-Blast can be used to perform an iterated search that detects distant relationship between molecules. See Altschul et al. (1997), supra. When utilizing BLAST, Gapped BLAST, and PSI-Blast programs, the default parameters of the respective programs (e.g., XBLAST and NBLAST) can be used. See ncbi.nlm.nih.gov/BLAST/ on the WorldWideWeb. Another non-limiting example of a mathematical algorithm utilized for the comparison of sequences is the algorithm of Myers and Miller, CABIOS 1988; 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932, 419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nuc. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### III. Antibody Drug Conjugates (ADC)

(A) Antibodies

Antibodies encompassed by the present invention may target any polypeptides involved in a proteinopathy. For example, amyloidogenic polypeptides involved in plaque deposits and fibril formation (e.g., Aβ), amyloid associated proteins (e.g. α₁-antichymotrypsin, Apolipoprotein E, apoE, and Creb Binding Proteins), tau, as well as prions, are suitable targets for antibodies according to the present invention. Other, non-limiting examples of antibody targets encompassed by the present invention, include for example, IAPP (Amylin), calcitonin, atrial natriuretic factor (AANF), apolipoprotein AI (AApoA1), serum amyloid A (AA), medin (AMed), transthyretin (ATTR), lysozyme (ALys), beta 2 microglobulin (Aβ2M), Gelsolin (AGel), keratoepithelin (AKer), beta amyloid (Aβ), cystatin (ACys), immunoglobulin light chain AL (AL), tau polypeptides, α-synuclein, Huntingtin, and superoxide dismutase. Further, suitable targets include whole protein and protein fragments and post-translational modifications, e.g., phosphorylated sites. See, Southwell and Patterson (2010) Reviews in the Neurosciences 21, 273-287.

An antibody according to the present invention may be targeted, for example, to the amino terminus or the carboxy terminus of the target polypeptide, or the antibody may be targeted to a mid domain of the target polypeptide. The antibody may also be a conformational antibody (i.e., may recognize a specific structure of the folded amyloid or amyloid associated polypeptide). The antibody may recognize any part or conformation of a target polypeptide. Non-limiting examples of antibodies specific for (targeted to) amyloid beta include, for example, bapineuzumab (see, http://www.alzforum.org/drg/drc/detail.asp?id=101), Ponezumab (see, http://www.alzforum.org/drg/drc/detail.asp?id=139), gantenerumab (see, http://www.alzforum.org/new/detail.asp?id=2933), solaneszumab, (see, http://www.alzforum.org/drg/drc/detail.asp?id=126) MABT5102A, (see, http://www.alzforum.org/drg/drc/detail.asp?id=121) GSK933766A (see, Cynthia A. Lernere & Eliezer Masliah Nature Reviews Neurology 6, 108-119 (February 2010)), IN-N01 (Yamaguchi et al., U.S. Pat. No. 7,807,157), 1C3 (Yamaguchi et al., U.S. application Ser. No. 12/888,661), 1A-10 (Yamaguchi et al., U.S. Pat. No. 7,807,157) and RN6G (Ding et al., 2011, Proc. Natl. Acad. Sci. 108(28) E279-E287).

Other non-limiting, specific examples of an amyloid beta targeted antibody is an exogenous free-end specific antibody which is targeted to a free N-terminus of amyloid β peptide or a free C-terminus of amyloid β peptide Aβ1-40. In another embodiment, an antibody is targeted to a C-terminus truncated amyloid β peptide fragment. In yet another embodiment, the antibody is targeted to a free N-terminus of amyloid β peptide or a free C-terminus of amyloid β peptide Aβ1-40. In another embodiment, the antibody is free-end specific and is targeted to the free C-terminus of the amyloid β-peptide Aβ1-39, Aβ1-40, Aβ1-41, or Aβ1-43. See, U.S. Patent Application Publication No. 2003/0073655 by Chain. In a specific embodiment, the antibody may be produced by hybridoma clone 82E1, which was deposited under the terms of the Budapest Treaty with the International Patent Organism Depository, National Institute of Advanced Industrial Science and Technology, AIST Tsu-kuba Central 6, 1-1, Higashi 1-chome Tsukuba-shi, Ibaraki-ken 305-8566, Japan on Feb. 3, 2010 and assigned accession no. FERM BP-11228. See, U.S. Pat. No. 7,807,157, where that antibody is characterized in detail.

The present invention also encompasses antibodies that are targeted to tau. Any region of the tau polypeptide may be a suitable target of the antibody. In a specific embodiment, an antibody may selectively recognize a phosphorylated form of tau. In another embodiment, an antibody may recognize an abnormal tau protein, such as a truncated form of tau or a phosphorylated abnormal tau protein, or may recognize a free end of a truncated abnormal tau protein. Non-limiting examples of antibodies that target tau, include, for example, PHF1 (which recognizes tau with phosphorylated serines 396 and 404 (see, Boutajangout et al. (2011) J. Neurochem; 1471-4159)); MC1 (a conformation dependent antibody that recognizes an early pathological tau conformation) (see, Chai et al. (2011) J Biolo Chem; 286:34457-34467), anti-tau (421/422) (see, Garcia-Sierra et al. (2003) J. Alzheimer' Disease 5(2) 65-77; Tau C3 (Signet, MBL, Invitrogen); tau dimer and oligomer selective TOC-1 (Patterson et al., J. Biol. Chem. 286: 23063-23076); TOMA antibodies, e.g., T2286 (International Patent Application WO/2011/026031); Anti-tau Glu 391, FITC-tagged halpha-syn antibody (see, Masliah et al (2005) Neuron; 46: 857-68); TauC3 (which recognizes tau when truncated at Asp421) (Gamblin et al., Proc Natl Acad Sci USA. 2003 August 19; 100(17): 10032-10037); 12E8 (which recognizes tau phosphorylated at Ser262) (Seubert P., et al., J Biol Chem. 270:18917, 1995; Letersky et al, Biochem. J., 1996); 1E1/A6 (deSilva R, Lashley T, et al. Neuropath & Applied Neurobio 29 (3):288, 2003); 8E6/C11 (deSilva R, Lashley T, et al. Neuropath & Applied Neurobio 29 (3):288, 2003); CP27 (Vingtdeux et al., Acta Neuropathol. 2011 March; 121(3):337-49; Herskovits et al., Neurobiol Dis. 2006 August; 23(2):398-408); CP9 (Roberson et al., Science. 2007 May 4; 316(5825):750-4); DA9 (Zempe et al., J Neurosci. 2010 Sep. 8; 30(36):11938-50); RTA-1 (Taniguchi T, Sumida M, et al., FEBS Lett. 579(6):1399-404); RTA-2 (Taniguchi T, Sumida M, et al., FEBS Lett. 579(6):1399-404); CP13 (which detects tau phosphorylated at Ser202); MC6 (which recognizes tau phosphorylated at Ser 235) (Jicha et al., J. Neurochem. 69, 2087-2095 (1997)); and 2E12 (highly specific for tau phosphorylated at Thr231) (Vingtdeux et al., Acta Neuropathol. 2011 March; 121(3):337-49). See also, U.S. Patent Application No. 61/438,083 by Chain, which describes the structure and sequence of tau and describes exemplary antibodies that can be used to target tau in an ADC according to the present invention.

Another target of an antibody of the invention is pathogenic prion PrPSc. Like amyloid fibrils, abnormal protein folding into β-sheet structures to form amyloid-like deposits is also widely believed to be the cause of prion-related encephalophathies, such as Creutzfeldt-Jakob disease (CJD) and Gerstmann-Straussler-Scheinker disease (GSS) in humans, scrapie in sheep and goats, and spongiform encephalopathy in cattle. The cellular prion protein (PrPc) is a sialoglycoprotein encoded by a gene that in humans is located on chromosome 20. The PrP gene is expressed in neural and non-neural tissues, the highest concentration of mRNA being in neurons. The translation product of PrP gene consists of 253 amino acids in humans, 254 in hamster and mice or 256 amino acids in sheep and undergoes several post-translational modifications. In hamsters, a signal peptide of 22 amino acids is cleaved at the N-terminus, 23 amino acids are removed from the C-terminus on addition of a glycosyl phosphatidylinositol (GPI) anchor, and asparagine-linked oligosaccharides are attached to residues 181 and 197 in a loop formed by a disulfide bond. In prion-related encephalopathies, PrPc is converted into an altered form designated PrPSc, that is distinguishable from PrPc in that PrPSc (1) aggregates; (2) is proteinase K resistant in that only the N-terminal 67 amino acids are removed by proteinase K digestion under conditions in which PrPSc is completely degraded; and (3) has an alteration in protein conformation from α-helical for PrPSc to an altered form.

Any antibody that recognizes pathogenic prion (e.g., misfolded prion protein, PrPSc) is contemplated for use in an ADC of the invention. Further, any region of a pathogenic prion may be a suitable target of the antibody. A specific example is ICSM 18 (see, Antonyuk et al. (2009) Proc. Natl. Acad Sci USA; 106(8): 2554-2558).

Anti-α-synuclein antibody therapy for the treatment of Parkinson's disease is reviewed in Southwell and Patterson, supra. Anti-α-synuclein antibodies are contemplated for use in the present invention. Examples are LB509 which detects C-terminal alpha synuclein (Baba et al., American Journal of Pathology, Vol. 152, No. 4, April 1998), Syn211 and Syn208 (Giasson et al., Neuron. 2002 May 16; 34(4):521-33) and 11A5, which detects α-synuclein phosphorylated at Serine 129 (Paleologou et al., Neurosci. 2010 Mar. 3; 30(9):3184-98).

Any antibody that recognizes pathogenic huntingtin protein, for example oligomers or cleavage products, is also contemplated for use in an ADC for this invention. Further, any region or posttranslational modification of a huntingtin protein may be suitable as a target of the antibody. Specific examples are MW1, which binds to the expanded polyQ repeat form of Htt, displaying no detectable binding to normal Htt (Ko et al., Brain Research Bulletin Volume 56, Issues 3-4, 1 Nov. 2001, Pages 319-329), EP867Y which is specific to the apopain cleavage site of human Huntingtin (Cho et al., Neuroscience. 2009 Nov. 10; 163(4):1128-34), and MAB2166 (Kaltenback, L. et al. (2007). PloS Genetics 3:0689-0708).

Antibodies of the present invention may also recognize α1-antichymotrypsin (e.g., alpha-1-Antichymotrypsin (ACT), Verdana Cat No 760-2604; Isaacson P, et al., (1979) Lancet. 2:964-965; Palmer P E, et al., (1974) Am J Clin Pathol 62:350-354; Palmer P E, et al., (1980) Cancer 45:1424-1431), apoE (ApoE4 Antibody (4E4) Novus Biologicals Cat No. NBP1-49529; Santa Cruz Biotechnology, Santa Cruz, Calif.) and Creb Binding Proteins (e.g., CBP (C-1): Cat No sc-7300).

In certain embodiments, an antibody according to the present invention may be an IgG4 or IgG2 antibody.

Antibodies according to the present invention may also be chimeric or humanized. Publications such as EP0125023, EP0239400, EP045126, WO94/20632, Protein Eng Des Sel. 2004 May; 17(5):481-489. Epub 2004 Aug. 17; Ann Oncol. 1998 May; 9(5):527-34; and Proc Natl Acad Sci USA. 1992 May 15; 89(10):4285-9, and the like can be referred to for preparation of chimeric antibody and humanized antibody. Briefly, to create a humanized antibody, the murine CDRs can be inserted into a human framework using methods known in the art. See, e.g., U.S. Pat. No. 5,225,539 to Winter, and U.S. Pat. Nos. 5,530,101; 5,585,089; 5,693,762 and 6,180,370 to Queen et al. Human antibodies can be generated by immunizing transgenic or transchromosomic mice in which the endogenous mouse immunoglobulin genes have been inactivated and exogenous human immunoglobulin genes have been introduced. Such mice are known in the art (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. Nos. 5,939,598; 6,075,181; 6,114,598; 6,150,584 and 6,162,963 to Kucherlapati et al; and PCT Publication WO 02/43478 to Ishida et al.) Human antibodies can also be prepared using phage display methods for screening libraries of human immunoglobulin genes. Such phage display methods for isolating human antibodies also are known in the art (see e.g., U.S. Pat. Nos. 5,223,409; 5,403,484; and 5,571,698 to Ladner et al; U.S. Pat. Nos. 5,427,908 and 5,580,717 to Dower et al; U.S. Pat. Nos. 5,969,108 and 6,172,197 to McCafferty et al; and U.S. Pat. Nos. 5,885,793; 6,521,404; 6,544,731; 6,555,313; 6,582,915 and 6,593,081 to Griffiths et al.

Also contemplated for use herein are camel antibodies. Camels generate functional antibodies consisting of only two heavy chains. These differ from those of conventional antibodies in that they lack the CH1 domain. Biophysical studies have revealed that camel antibodies have a number of unique features when compared with those of conventional antibody molecules, notably their smaller size, greater solubility and higher stability See, Tayebi et al. (2010) Journal of General Virology, 91, 2121-2131. Camel antibodies are capable of binding to intracellular targets. Non-limiting of prion specific camel antibodies are described in Tayebi et al, supra, however, the skilled artisan that camel antibodies may be directed against any of the other targets described herein (e.g., amyloid beta, tau, apoE, etc.).

Antibodies of the invention may contain one or more amino acid mutations, such as, but not limited to, mutations in the framework regions, or even within the complementarity determining regions (CDRs). Preferably, a mutation in the CDR does not alter, or enhances, binding of the antibody to its target, compared to the unmutated antibody.

Another type of framework modification involves mutating one or more residues within the framework region or even within one or more CDRs, to remove T cell epitopes to thereby reduce the potential immunogenicity of the antibody. This approach is also referred to as “deimmunization” and is described in further detail in U.S. Patent Publication No. 2003/0153043 by Can et al.

In addition or in alternative to modifications made within the framework or CDRs, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH₂—CH₃ domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, to increase the biological half life, the antibody can be altered within the CH₁ or C_{L} region to contain a salvage receptor binding epitope taken from two loops of a CH2 domain of an Fc region of an IgG, as described in U.S. Pat. Nos. 5,869,046 and 6,121,022 by Presta et al.

In still another embodiment, the C-terminal end of an antibody of the present invention is modified by the introduction of a cysteine residue as is described in detail in WO 2009/026274. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In certain embodiments, the cysteine-containing extension comprises the sequence alanine-alanine-cysteine (from N-terminal to C-terminal).

In certain embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al., Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha(1,6)fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al, and PCT Publication No. WO/2007/055916 to Ravetch et al. For example, one may employ an enzymatic reaction with sialidase, such as, for example, Arthrobacter ureafacens sialidase. The conditions of such a reaction are generally described in the U.S. Pat. No. 5,831,077. Other non-limiting examples of suitable enzymes are neuraminidase and N-Glycosidase F, as described in Schloemer et al., J. Virology, 15(4), 882-893 (1975) and in Leibiger et al., Biochem J., 338, 529-538 (1999), respectively. Desialylated antibodies may be further purified by using affinity chromatography.

Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

(B) Partner Molecules

The ADC-containing compositions provided herein comprise an antibody conjugated to a partner molecule. In certain embodiments, the partner molecule is a small molecule. In particular, the small molecule is a cytoprotective agent, such as, but not limited to, an antioxidant, an antifibrillogenic molecule, or an insulin sensitizing molecule. Non-limiting examples of antioxidants according to the present invention include melatonin, an indole amine, an indole acid (such as indole-3-propionic acid, and other indole acids such as pyruvic and acetic butyric), vitamin E, vitamin C, lipoic acid, uric acid, circumin, glutathione, a polyphenol, a flavonoid, an anthraquinone methylthioninium chloride, dimebone, a rhodanine-based compounds, an insulin sensitizer e.g. pioglitazone and rosiglitazone, an 8-hydroxyquinolone derivative e.g. PBT2, PBT434, penicillamine, trientine, a tetracycline, (N-(pyridin-2-ylmethyl)aniline), N1,N1-dimethyl-N4-(pyridin-2-ylmethyl)benzene-1,4-diamine, 2,6-diaminopyridine, AZD-103, cyclohexane-1,2,3,4,5,6-hexyl, myo-inositol, scyllo-inositol, indole-3-propionic acid, (N-(pyridin-2-ylmethyl)aniline), methylene blue, TRx0014, and NO scavengers.

Examples of partner molecules include melatonin and melatonin analogs that retain the antioxidant properties of melatonin, and preferably, although not necessarily, the ability to interact with the high affinity copper binding site in Aβ and to inhibit fibril formation.

Analogs of melatonin include compounds that interact with melatonergic systems, for example, compounds that interact with the melatonin receptor. Many examples of such compounds are known in the art. See, for example, U.S. Pat. Nos. 5,449,690, 5,464,872, 5,470,846, 5,541,228, 5,552,418, 5,552,428, 5,554,642, 5,580,878, and 5,591,775. Melatonin analogs can readily be assayed to ensure that the antioxidant function of melatonin is retained, using the methodology disclosed herein, such as assays for cell viability, lipid peroxidation, intracellular Ca²⁺, and oxygen free-radicals. The prevention of other cytotoxic effects of Aβ on cells can readily be observed microscopically, such as the prevention of membrane blebbing, cell retraction, abnormal distribution of chromatin, and karyorrhexis. As indicated above, the cytotoxic or cell killing effects of Aβ include, for example, decreased cell viability (i.e. cell death), increased lipid peroxidation (an indicator of increased oxygen free-radicals), increased intracellular Ca²⁺, levels, diffuse membrane blebbing, cell retraction, abnormal distribution of chromatin towards the nuclear membrane, and karyorrhexis. The cytotoxic effects of Aβ are most readily seen in neuronal cells (including cells of the central and peripheral nervous systems), and occur in human subjects afflicted with AD. It may also be determined whether the analogue maintains the ability to interact with the high affinity copper binding site in amyloid beta.

Among possible neuroprotective agents, frontline candidates are those that prevent protein aggregation, inhibit accumulation of reactive oxygen species or prevent other processes before irreversible damage to nerve cells has occurred. Neuroprotective approaches to the treatment of theses diseases can include free radical scavengers, especially hydroxyl radical scavengers, nitrous oxide scavengers, selective metal chelators, metal attenuating compounds, electron transfer stimulators, and anti-inflammatory compounds.

The endogenous hormone melatonin holds promise as a neuroprotective compound for the treatment of neurodegenerative disease and its age related decline might also contribute to increased levels of oxidative stress in the elderly. Melatonin is a potent antioxidant consuming 4 moles of hydroxyl radical per mole of melatonin. The compound has also been shown to bind to amyloid beta and to inhibit the progressive formation of βsheets and fibrils. Melatonin also has multiple actions as a regulator of antioxidant and prooxidant enzymes, radical scavenger and antagonist of mitochondrial radical formation. Moreover, melatonin is incapable of forming damaging pro-oxidant intermediates upon metabolism. The ability of melatonin and its kynuramine metabolites to interact directly with the electron transport chain by increasing the electron flow and reducing electron leakage are unique features by which melatonin is able to increase the survival of neurons under enhanced oxidative stress. Moreover, antifibrillogenic actions of melatonin have been demonstrated in vitro, also in the presence of profibrillogenic apoE4 or apoE3, and in vivo, in a transgenic mouse model. Amyloid beta toxicity is prevented by melatonin and analogues such as indole-3-propionic acid in vitro models. Melatonin treatment has been also demonstrated to be beneficial in animal models of AD, PD and HD and to protect retinal epithelial cells in models of AMD. However, melatonin has had modest benefit affecting disease progress in human clinical trials of AD except by improving sleep disturbances based on its receptor-mediated regulation of circadian rhythms at low doses of the hormone. At higher doses, such as for neuroprotection, melatonin may be expected to result in adverse off-target effects. According to the Mayo Clinic (http://www.mayoclinic.com/health/melatonin-side-effects/AN01717), melatonin side effects may include daytime sleepiness, dizziness, headaches, abdominal discomfort, confusion, sleepwalking, and nightmares. Melatonin may also interact with various medications, including blood-thinning medications (anticoagulants), immunosuppressants and diabetes medications. It would be desirable to prevent some of the side effect of melatonin, such as by preventing binding of melatonin to its receptor following administration to a subject.

A major limitation of current melatonin based therapies for the treatment of proteinopathies is that it has an extremely short half life in the blood and in the eye which leads to poor bioavailability and the need for potentially harmful high doses administered chronically over many years. It would be desirable to increase the half-life of melatonin and thus widen the therapeutic window.

In human antioxidant studies, Vitamin E (α-tocopherol), a potent chain breaking antioxidant, is one of the most extensively studies antioxidant agents. Data from cross-sectional and longitudinal studies assessing the relationship between Vitamin E consumption and AD risk have led to conflicting resulted while high levels of Vitamin consumption over time in the general population has been associated with increased morbidity. The most promising clinical trial to date was conducted with Vitamin E supplementation of 2000 IU per day for moderate-stage AD patients which led to a small but significant delay in reaching the end points of institutionalization, loss of major activities of daily living, or death, but did not delay loss of cognitive performance.

A major limitation of Vitamin E as a potential therapy for AD or other proteinopathies affecting the brain is that even a massive increase in vitamin E intake does not result in an increase of this vitamin's concentration in the brain because of its poor BBB permeation and tight regulation within the brain. In contrast, liver and muscle are well-receptive of rising Vitamin E supplies. It would be desirable to increase the bioavailability of Vitamin E in the brain without altering its antioxidant activity and deliver concentrated localized concentrations of the drug to the sites of amyloidosis, preferably together with an agent that promotes amyloid clearance.

There is substantial in-vitro data indicating that Curcumin has antioxidant, anti-inflammatory, and anti-amyloid activity. In addition, studies in AD animal models of indicate a direct effect of Curcumin in decreasing the amyloid pathology of AD. As the widespread use of Curcumin as a food additive and relatively small short-term studies in humans suggest safety, Curcumin is a promising agent in the treatment and/or prevention of AD.

Two major limitations of Curcumin as a therapy for AD are first, that it has very poor penetration into the brain and secondly, that it is rapidly eliminated from the body. It would be desirable to increase the penetration of Curcumin into the brain, increase brain retention, deliver high, localized concentrations to sites of oxidative stress caused by amyloid proteins and reduce its elimination from the blood, preferably together with an agent that promotes amyloid clearance.

Cu2+ and Zn2+ promote aggregation of Aβ and chelation of these metals renders Aβ aggregates less compact and less resistant turnover. Derivatives of 8-Hydroxyquinoline are being developed as potential therapeutics for proteinopathies based on the metal attenuating properties. PBT2 is a copper/zinc ionophore that rapidly restores cognition in AD mouse models and also shown beneficial effects in HD models. A recent Phase IIa double-blind, randomized, placebo-controlled trial found that the 250 mg dose of PBT2 was well-tolerated, significantly lowered cerebrospinal fluid (CSF) levels of amyloid-beta42, and significantly improved executive function on a Neuro-psychological Test Battery (NTB) within 12 weeks of treatment in patients with AD. PBT434 has been shown that it is able to impede the iron-induced oxidative damage and neurotoxic cascade that kills the substantia nigra in PD. Other metal chelating compounds of interest include trientine, penicillamine, 2,6-diaminopyridine.

A limitation of metal binding molecules is the possibility of interfering with non-pathogenic metal dependent enzymes and other physiological processes. It would be desirable to be able to deliver high, localized concentrations of PBT2 and PBT434 in the vicinity of amyloidogenic metal bound proteins such as Aβ, alpha synuclein and huntingtin, preferably together with an agent that promotes amyloid clearance.

Rhodanine-based compounds are frequently employed in medicinal chemistry with no apparent side effects and the core has been investigated for tau aggregation inhibition via the synthesis of a focused library. Rhodanine heterocycle was the most effect compound with an IC50 of 0.8 μM representing the assembly inhibiting half maximal concentration measured in vitro and an DC50 of 0.1 μM in relation to disassembling-inducing activity. The rhodanine heterocycle appeared to contribute the main activity in each case. Besides the central rhodanine core, hydrogen bond acceptors in the form of a nitro group, carboxylic acids, phenols, sulfonates/sulfonamides are required in line with observations from other known amyloid inhibitors. Noteworthy, the total length of the molecule proved to be of importance. Variations of the length of the linker between the carboxylic acid and the rhodanine core revealed that increasing the distance up to two carbons bonds resulted in an appreciable increase in the compound's inhibitory potency indicating the optimal positioning of the inhibitor toward its bindings site. An example of a rhodanine compound suitable for this invention is pioglitazone. The mechanism for development of AD has been linked to both inflammation and decreased insulin sensitivity. Because of this, pioglitazone has been evaluated as potential treatment for AD because of its insulin-sensitizing and anti-inflammatory effects.

A significant limitation of rhodanine-based compounds as therapeutic compounds for treating AD and other tauopathies is that relative potencies in vitro are not well correlated in vivo because of various ADME parameters (adsorption, distribution, metabolism and excretion indicating that molecules need to be carefully optimized. It would be desirable to select compounds based on potency determine from in vitro assays without need for further optimization.

Numerous polyphenols show inhibitory activity in a variety of amyloids such as alpha synuclein, IAPP, Aβ, PrPsc. Mycetin has been reported as tau aggregation inhibitors with a 1.2 μM IC₅₀ and the in vivo data indicated with the elongation phase of the fibril assembly.

A major limitation of dietary polyphenols is due to lack of bioavailability including poor uptake across the intestine followed by extensive metabolism on reaching the plasma through methylation, sulfation and glucuronidation. Moreover, while penetration across the blood brain barrier is thought to be poor, high brain concentrations of polyphenols pose significant safety concerns with increasing evidence of acute polyphenol toxicity at high doses. It would be desirable to have a chaperone to improve the bioavailability of polyphenols while concentrating their neuroprotective function to sites of amyloidosis.

Solid-state nuclear magnetic resonance (ssNMR) may be used to identify key residues within amyloidogenic protein sequences that may be targeted to inhibit the aggregation of the host protein. For α-synuclein, the major protein component of Lewy bodies associated with Parkinson's disease, combination of ssNMR and biochemical data was used to identify the key region for self-aggregation of the protein as residues 77-82 (VAQKTV) which led to the design of a new peptide derived from residues 77 to 82 of α-synuclein with an N-methyl group at the C-terminal residue, which was able to disrupt the aggregation of α-synuclein.

A major limitation of using short peptides as therapeutic for the treatment of proteinopathies is that they are rapidly eliminated from the body. It would be desirable to increase the bioavailability of short peptide inhibitors of protein aggregation and increase retention in the brain

LMTX™, is a second-generation tau aggregation inhibitor containing the active ingredient, methylthioninium (MT). The LMTX™ family also has activity against synuclein aggregation. LMTX activity could be synergistic if applied with other therapies for example an antibody against either tau protein.

Suitable assays for determining whether a melatonin analogue maintains one or more properties or functions of melatonin (e.g., an ability to interact with amyloid beta peptide and/or to inhibit fibril formation) are described in detail in Pappolla et al. (1997) J Biol Chem 273(13):7185-7188. See, also, Cheng et al. (2005) Anal. Chem. 2005, 77, 7012-7015, which describes a mass spectrometry-based screening assay for identifying compounds that inhibit the aggregation of Aβ.

Oxidative damage has been suggested to be the primary cause of aging and age-associated neurodegenerative diseases like AD, PD, and HD. This concept is based on the free radical hypothesis of aging as proposed by Harman. See, Harman D: (1956). J Gerontol; 11:298-300. There is compelling evidence for a decisive participation of severe oxidative stress in the development of neuropathology seen in AD Immunohistochemical studies confirmed findings demonstrating increased levels of lipid peroxidation in vitro observed in autopsy samples of brains afflicted by AD. Because of its high rate of oxygen consumption and its high content of polyunsaturated fatty acids, the brain exhibits increased vulnerability to oxidative stress. Elevated lipid peroxidation, as found in the brains of AD patients, not only reveals oxidative stress, but also exerts secondary effects on protein modification, oxidation and conformation.

Increased protein and DNA oxidation also occurs in AD. Measurements of protein carbonyl, 3,3′-dityrosine and 3-nitrotyrosine in post mortem brain samples from AD patients have shown increased oxidative and nitrosative protein modification in the hippocampal and neocortical regions, but not in the cerebellum. Free radical attack on DNA results in strand breaks, DNA-protein cross linkage, and base modification. Double- and single-strand breaks were elevated in AD cortex and hippocampus, but this has to be largely attributed to apoptotic fragmentation. Enhanced oxidative DNA modification is, however, also demonstrable, mostly as 8-hydroxy-2′-deoxyguanosine (8-OHdG), a product primarily formed by attack of hydroxyl radicals, but other modified bases such as 8-OH-adenine have also been demonstrated.

Augmented free radical damage to lipids, proteins and nucleic acids has been reported for the substantia nigra of parkinsonian patients. Therefore, numerous compounds with antioxidant properties have been suggested for treatment of AD and other neurodegenerative diseases. Among these substances, melatonin is unique for several reasons: it is a natural compound synthesized in the pineal gland and other body tissues; it can be released by the pineal gland via the pineal recess into the cerebrospinal fluid (CSF), in much higher concentrations than into the circulation; its production decreases with the advancement of age, a fact which has been suggested to be one of the major causes of age-associated neurodegenerative diseases. For a comprehensive review of melatonin, see Srinivasan et al. (2006) Behavioral and Brain Functions, 2:15.

A partner molecule, such as, e.g., an antioxidant, may also be modified, e.g., to further increase bioavailability. For example, an antioxidant may be chemically modified with a fluorinated amphiphilic carrier to increase bioavailability and/or half-life in vivo. See, Ortial et al. (2006) J Med Chem. 49(9):2812-20.

A partner molecule may also be labeled with a marker. Labels are useful for enabling, e.g., in vivo imaging of a site targeted by an ADC of the invention. (e.g., amyloid deposit and/or neurofibrillary tangle). For example, and without limitation, a marker can be any label that generates a detectable signal, such as a radiolabel, a fluorescent label (e.g., GFP), or an enzyme that catalyzes a detectable modification to a substrate (e.g., horseradish peroxidase).

Specific examples of how partner molecules can be conjugated are given below: In certain cases, these small molecules (or “payloads”) could be conjugated via use of non-cleavable linkers through available functional groups, for example, hydroxyl or thiol groups, whereas in other cases, the payload may require modification before it can be conjugated either to a cleavable or non-cleavable linker. For example, the phenolic OH group is available as a reactive group in tocopherol and curcumin and could be used in combination with a non-cleavable linker. However, it would be important to determine if the free radical scavenging properties of tocopherol or curcumin are inhibited in this way, in which case it may be preferable to use cleavable linkers as described in WO 2007089149A2 or J. Med. Chem. 2005, 48, 1344-1358. The same is true for derivatives of 8-Hydroxyquinoline such as PBT2 or PBT434 and with respect to the OH group in indole-3-propionic acid. Since both the neurogenesis enhancers P7C3A20 and P7C3 are active, it may be preferable to conjugate P7C3, which has an OH group. For penicillamine, one might use SMCC linker (e.g. T-DM1) or similar for conjugation via the free thiol group or use the primary amine in the context of cleavable linkers. Similarly, one can make use of the primary amines in 2,6-diaminopyridine and its derivatives and similarly with trientine. In view of the absence of available free functional groups in molecules such pioglitazone, TRx0014 and Ponatinib, for example, these drugs would need to be modified before they could be conjugated. Table 1 provides a list of partner molecules of the invention with possible antibody combinations and linker types.

TABLE 1
Example Antibody Conjugates of the Invention

| Partner Molecule | Partner Molecule Structure | Antibody | Linker |
|---|---|---|---|
| Melatonin | 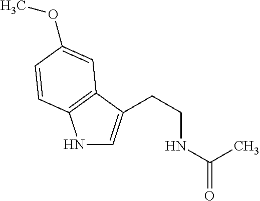 [SMILES: COc1ccc2[nH]cc(CCNC(C)=O)c2c1] | Amyloid beta | Non-cleavable |
| alpha tocopherol | 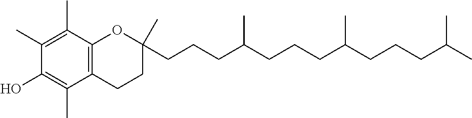 [SMILES: Cc1c(C)c2c(c(C)c1O)CCC(C)(CCCC(C)CCCC(C)CCCC(C)C)O2] | Amyloid beta | Non-cleavable |
| 8- Hydroxyquinoline derivative e.g. PBT434 | 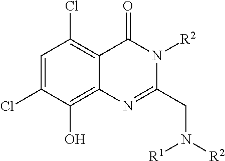 | alpha synuclein | Cleavable |
| REMBER TRx0014 | 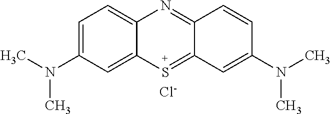 | Tau | Cleavable/non- cleavable |
| Rhodanine derivatives | 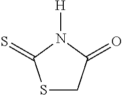 | Tau | Non-cleavable |
| phenylthiazolyl- | | Tau | Non-cleavable |
| hydrazide | | | |
| derivatives | | | |
| Pioglitazone | 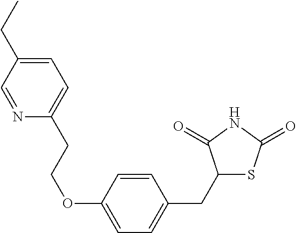 [SMILES: CCc1ccc(CCOc2ccc(CC3SC(=O)NC3=O)cc2)nc1] | amyloid beta, tau, alpha synuclein & huntingtin | Cleavable |

Antibody-drug conjugates have more complex and heterogenous structures than the corresponding antibodies. The selection of the most appropriate methods for a specific ADC is heavily dependent on the properties of the linker, the drug and the choice of attachment sites (lysines, interchain cysteines, Fc glycans). Improvements in analytical techniques such as protein mass spectrometry and capillary electrophoresis have significantly increased the quality of information that can be obtained for use in product and process characterization and for routine lot release and stability testing necessary to ensure that they are optimized for use in pharmacological compositions. Methods and approaches are reviewed in Wakankar et al., 2011, mAbs 3:2 161-172 (Landes Bioscience).

(C) Linkers

In some embodiments of the invention, an antibody is conjugated directly to a partner molecule (e.g., cytoprotective agent or other small molecule) via a cysteine residue at or near the C-terminus of a heavy chain of the antibody. The cysteine residue may be naturally occurring in the heavy chain or, in certain embodiments, the cysteine residue at the C-terminus of the heavy chain is introduced by the replacement of the original C-terminal amino acid residue.

In other embodiments, a partner molecule is conjugated to an antibody by a chemical linker (sometimes referred to herein simply as “linker”).

The ratio of partner molecules attached to an antibody can vary, depending on factors such as the amount of partner molecule employed during conjugation reaction and the experimental conditions. Preferably, the ratio of partner molecules to antibody is between 1 and 20, more preferably between 1 and 10. Those skilled in the art will appreciate that, while each individual molecule of antibody Z is conjugated to an integer number of partner molecules, a preparation of the conjugate may analyze for a non-integer ratio of partner molecules to antibody, reflecting a statistical average.

Linkers inherently have shorter half-lives than their antibody counterparts, and therefore typically, although not necessarily, need to be modified to improve their solubility. This can be accomplished, for example, by conjugating a polyethylene glycol (PEG), or PEG-like derivative, to the linker. However, it will be understood that other modifications are known to those of skill in the art and can be used in the ADC described herein.

In some embodiments, the linker is a peptidyl linker, depicted herein as (L⁴)ₚ-F-(L¹)ₘ. Other linkers include hydrazine and disulfide linkers, depicted herein as (L⁴)ₚ-H-(L¹)ₘ, and (L⁴)ₚ-J-(L¹)ₘ, respectively. F, H, and J are peptidyl, hydrazine, and disulfide moieties, respectively, that are cleavable to release the partner molecule from the antibody, while L¹ and L⁴ are linker groups. F, H, J, L¹, and L⁴ are more fully defined herein below, along with the subscripts p and m. The preparation and use of these and other linkers are described in detail in WO 2005/112919.

The use of peptidyl and other linkers in ADC is described in U.S. Pat. Application Pub. No. 2006/0004081; 2006/0024317; 2006/0247295; U.S. Pat. No. 6,989,452; U.S. Pat. No. 7,087,600; and U.S. Pat. No. 7,129,261; WO 2007/051081; WO 2007/038658; WO 2007/059404; and WO 2007/089100. Additional linkers are described in U.S. Pat. No. 6,214,345; and U.S. Pat. Application Pub. No. 2003/0096743; and 2003/0130189; in de Groot et al., J. Med. Chem. 42, 5277 (1999); de Groot et al. J. Org. Chem. 43, 3093 (2000); de Groot et al., J. Med. Chem. 66, 8815, (2001); WO 02/083180; Carl et al., J. Med. Chem. Lett. 24, 479, (1981); Dubowchik et al., Bioorg & Med. Chem. Lett. 8, 3347 (1998).

In addition to connecting the antibody and the partner molecule, a linker can impart stability to the partner molecule, reduce its in vivo toxicity, or otherwise favorably affect its pharmacokinetics, bioavailability and/or pharmacodynamics. It is generally preferred that the linker is cleaved, releasing the partner molecule, once the conjugate is delivered to its site of action. Also preferably, the linkers are traceless, such that once cleaved, no trace of the linker's presence remains.

In another embodiment, the linkers are characterized by their ability to be cleaved at a site in or near a target cell such as at the site of therapeutic action or marker activity of the partner molecule. Such cleavage can be enzymatic in nature. This feature aids in reducing systemic activation of the partner molecule, reducing toxicity and systemic side effects. Preferred cleavable groups for enzymatic cleavage include peptide bonds, ester linkages, and disulfide linkages, such as the aforementioned F, H, and J moieties. In other embodiments, the linkers are sensitive to pH and are cleaved through changes in pH. For example, a cleavable linker may be preferably hydrolysable at a pH of less than 5.5.

One aspect is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. WO 2005/112919 discloses hydrazine linkers that can be designed to cleave at a range of speeds, from very fast to very slow.

Linkers can serve to stabilize the partner molecule against degradation while the conjugate is in circulation, before it reaches the target tissue or cell. This is a significant benefit since it prolongs the circulation half-life of the partner molecule. A preferred partner molecule of the invention, melatonin, for example, has a particularly short half-life in blood. In some embodiments, the linker can serve to attenuate the activity of the partner molecule so that the conjugate is relatively benign while in circulation but the partner molecule has the desired effect, e.g., protects cells from oxidotoxins, after activation at the desired site of action. For therapeutic agent conjugates, this feature of the linker serves to improve the therapeutic index of the agent.

In addition to the cleavable peptide, hydrazine, or disulfide groups F, H, or J, respectively, one or more linker groups L¹ are optionally introduced between the partner molecule and F, H, or J, as the case may be. These linker groups L¹ may also be described as spacer groups and contain at least two functional groups. Depending on the value of the subscript m (i.e., the number of L¹ groups present) and the location of a particular group L¹, a chemical functionality of a group L¹ can bond to a chemical functionality of the partner molecule, of F, H or J, as the case may be, or of another linker group L¹ (if more than one L¹ is present). Examples of suitable chemical functionalities for spacer groups L¹ include hydroxy, carbonyl, carboxy, amino, ketone, aldehyde, and mercapto groups.

The linkers L¹ can be a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a PEG moiety.

Exemplary groups L¹ include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, aminal esters, nucleic acids, peptides and the like.

One function of the groups L¹ is to provide spatial separation between F, H or J, as the case may be, and the partner molecule, lest the latter interfere (e.g., via steric or electronic effects) with cleavage chemistry at F, H, or J. The groups L¹ also can serve to introduce additional molecular mass and chemical functionality into conjugate. Generally, the additional mass and functionality affects the serum half-life and other properties of the conjugate. Thus, through careful selection of spacer groups, conjugates with a range of serum half-lives can be produced. Optionally, one or more linkers L¹ can be a self-immolative group, as described herein below.

The subscript m is an integer selected from 0, 1, 2, 3, 4, 5, and 6. When multiple L¹ groups are present, they can be the same or different.

L⁴ is a linker moiety that provides spatial separation between F, H, or J, as the case may be, and the antibody, lest F, H, or J interfere with the antigen binding by the antibody or the antibody interfere with the cleavage chemistry at F, H, or J. Preferably, L⁴ imparts increased solubility or decreased aggregation properties to conjugates utilizing a linker that contains the moiety or modifies the hydrolysis rate of the conjugate. As in the case of L¹, L⁴ optionally is a self immolative group. In one embodiment, L⁴ is substituted alkyl, unsubstituted alkyl, substituted aryl, unsubstituted aryl, substituted heteroalkyl, or unsubstituted heteroalkyl, any of which may be straight, branched, or cyclic. The substitutions can be, for example, a lower (C₁-C₆) alkyl, alkoxy, alkylthio, alkylamino, or dialkyl-amino. In certain embodiments, L⁴ comprises a non-cyclic moiety. In another embodiment, L⁴ comprises a positively or negatively charged amino acid polymer, such as polylysine or polyarginine, L⁴ can comprise a polymer such as a PEG moiety. Additionally, L⁴ can comprise, for example, both a polymer component and a small molecule moiety.

In a preferred embodiment, L⁴ comprises a PEG moiety. The PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

The subscript p is 0 or 1; that is, the presence of L⁴ is optional. Where present, L⁴ has at least two functional groups, with one functional group binding to a chemical functionality in F, H, or J, as the case may be, and the other functional group binding to the antibody. Examples of suitable chemical functionalities of groups L⁴ include hydroxy, carbonyl, carboxy, amino, ketone, aldehyde, and mercapto groups. As antibodies typically are conjugated via sulfhydryl groups (e.g., from unoxidized cysteine residues, the addition of sulfhydryl-containing extensions to lysine residues with iminothiolane, or the reduction of disulfide bridges), amino groups (e.g., from lysine residues), aldehyde groups (e.g., from oxidation of glycoside side chains), or hydroxyl groups (e.g., from serine residues), preferred chemical functionalities for attachment to the antibody are those reactive with the foregoing groups, examples being maleimide, sulfhydryl, aldehyde, hydrazine, semicarbazide, and carboxyl groups. The combination of a sulfhydryl group on the antibody and a maleimide group on L⁴ is preferred.

In some embodiments, L⁴ comprises:

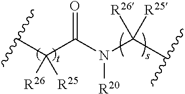

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²⁰, R²⁵, R^{25′}, R²⁶ and R^{26′} are hydrophobic. In some embodiments, R²⁰ is H or alkyl (preferably, unsubstituted lower alkyl). In some embodiments, R²⁰, R²⁵, R^{25′}, R²⁶ and R^{26′} are independently H or alkyl (preferably, unsubstituted C₁ to C₄ alkyl). In some embodiments, R²⁰, R²⁵, R^{25′}, R²⁶ and R^{26′} are all H. In some embodiments, t is 1 and s is 1 or 2.

Linker moieties of the invention also include, e.g., thioether linkers derived from maleimide derivatives. For example, maleimides are known to react with sulfhydryl groups at pH 6.5-7.5 to form stable thioether bonds. In particular, the compound SMCC (N-succinimidyl-4-(N-maleimidomethyl)-cyclohexane-1-carboxylate), having the structure:

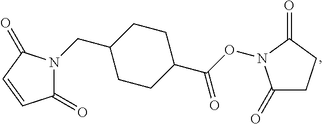 [SMILES: O=C(ON1C(=O)CCC1=O)C1CCC(CN2C(=O)C=CC2=O)CC1]

contains a sulfhydryl-reactive maleimide group to form a stable thioether bond. SMCC also contains an amine-reactive N-hydroxysuccinimide ester which reacts with primary amines. This compound is therefore well suited as a cross-linking agent for direct conjugation of antibodies to other functional moieties, such as partner molecules. In some embodiments, for example, the ADC is an antifibrillogenic molecule attached by an SMCC linker that is extended by a short PEG motif to increase the spatial separation between the partner molecule and the antibody backbone. See, Vollhardt and Schore, Organic Chemistry: Structure and Function, Fourth Edition, W. H. Freeman & Co. 0 2002 W. H. Freeman & Co., and Sumanas, Inc.

(i) Peptide Linkers (F)

As discussed above, the peptidyl linkers of the invention can be represented by the general formula: ((L⁴)ₚ-F-(L¹)ₘ, wherein F represents the portion comprising the peptidyl moiety. In one embodiment, the F portion comprises an optional additional self-immolative linker L² and a carbonyl group, corresponding to a conjugate of formula (a):

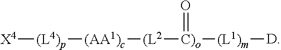

In this embodiment, L¹, L⁴, p, and m are as defined above. X⁴ is an antibody and D is a partner molecule. The subscript o is 0 or 1 and L², if present, represents a self-immolative linker. AA¹ represents one or more natural amino acids, and/or unnatural α-amino acids; c is an integer from 1 and 20. In some embodiments, c is in the range of 2 to 5 or c is 2 or 3.

In formula (a), (AA¹) is linked, at its amino terminus, either directly to L⁴ or, when L⁴ is absent, directly to X⁴. In some embodiments, when L⁴ is present, L⁴ does not comprise a carboxylic acyl group directly attached to the N-terminus of (AA¹)_{c}.

In another embodiment, the F portion comprises an amino group and an optional spacer group L³ and L¹ is absent (i.e., m is 0), corresponding to a conjugate of formula (b):

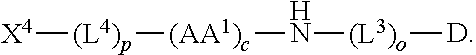

In this embodiment, X⁴, D, L⁴, (AA¹)_{c}, and p are as defined above. The subscript o is 0 or 1. L³, if present, is a spacer group comprising a primary or secondary amine or a carboxyl functional group, and either the amine of L³ forms an amide bond with a pendant carboxyl functional group of D or the carboxyl of L³ forms an amide bond with a pendant amine functional group of D.

(ii) Self-Immolative Linkers

A self-immolative linker is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleaved by such target enzyme at the bond covalently linking the spacer moiety and the peptide moiety to thereby effect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby effect release of the drug in pharmacologically active form. See, for example, Carl et al., J. Med. Chem., 24 (3), 479-480 (1981); WO 81/01145; Toki et al., J. Org. Chem. 67, 1866-1872 (2002); WO 2005/112919; and WO 2007/038658. See, also, U.S. Pat. No. 7,375,078.

One particularly preferred self-immolative spacer may be represented by the formula (c):

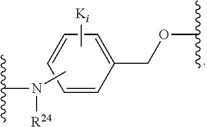

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, intro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH3, N(CH₃)₂, NHCOCF₃ and methyl. For “Kᵢ”, i is an integer of 0, 1, 2, 3, or 4. In one preferred embodiment, i is 0.

The ether oxygen atom of the above structure is connected to a carbonyl group (not shown). The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

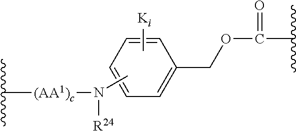

where R²⁴, (AA¹), K, and i are as defined above.

In another embodiment, the peptide linker of formula (a) above comprises a —F-(L¹)ₘ- that comprises the structure:

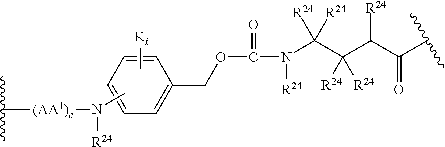

where R²⁴, (AA¹), K and i are as defined above.

In some embodiments, a self-immolative spacer L¹ or L² includes

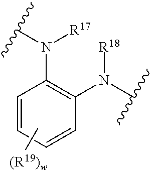

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹² and R¹⁸ are independently H or alkyl (preferably, unsubstituted C₁-C₄ alkyl). Preferably, R¹² and R¹⁸ are C₁₋₄ alkyl, such as methyl or ethyl. In some embodiments, w is 0. It has been found experimentally that this particular self-immolative spacer cyclizes relatively quickly.

In some embodiments, L¹ or L² includes

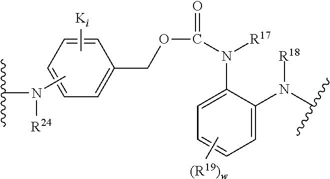

where R¹⁷, R¹⁸, R¹⁹, R²⁴, and K are as defined above.

(iii) Spacer Groups

The spacer group L³ comprises a primary or secondary amine or a carboxyl functional group, and either the amine of L³ forms an amide bond with a pendant carboxyl functional group of D or the carboxyl of L³ forms an amide bond with a pendant amine functional group of D. L³ can be selected from the group consisting of substituted or unsubstituted alkyl, substituted or unsubstituted heteroalkyl, substituted or unsubstituted aryl, substituted or unsubstituted heteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

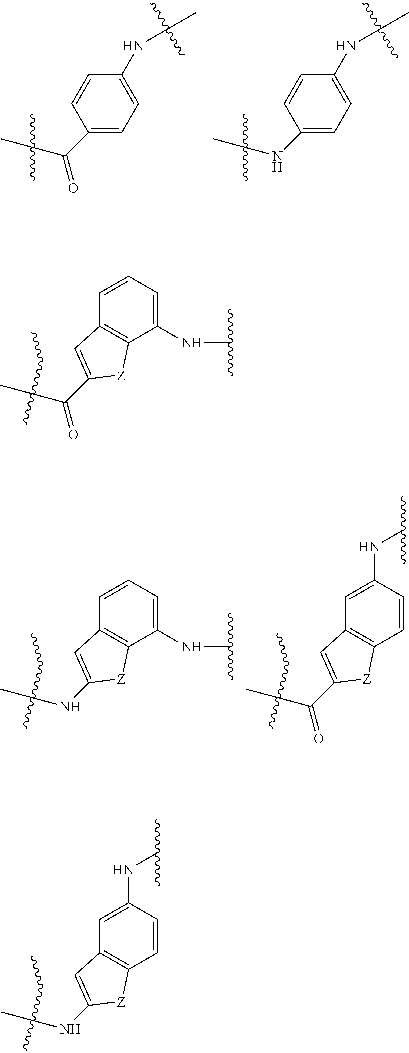

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its attachment to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

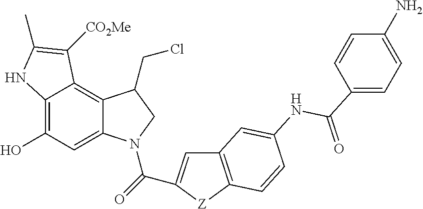

where Z is O, S or NR²³, where R²³ is H, substituted or unsubstituted alkyl, substituted or unsubstituted heteroalkyl, or acyl; and the NH₂ group on each structure reacts with (AA¹), to form -(AA¹)_{c}-NH—.

(iv) Peptide Sequence (AA¹)_{c}

The group AA¹ represents a single amino acid or a plurality of amino acids joined together by amide bonds. The amino acids may be natural amino acids and/or unnatural α-amino acids. They may be in the L or the D configuration. In one embodiment, at least three different amino acids are used. In another embodiment, only two amino acids are used.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, citrulline, and O-phosphoserine Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium Such analogs have modified R groups (e.g., norleucine) or modified peptide backbones, but retain the same basic chemical structure as a naturally occurring amino acid. One amino acid that may be used in particular is citrulline, which is a precursor to arginine and is involved in the formation of urea in the liver. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When attached to a linker or conjugate of the invention, the amino acid is in the form of an “amino acid side chain”, where the carboxylic acid group of the amino acid has been replaced with a keto (C(O)) group. Thus, for example, an alanine side chain is —C(O)—CH(NH₂)—CH₃, and so forth.

The peptide sequence (AA¹)_{c} is functionally the amidification residue of a single amino acid (when c=1) or a plurality of amino acids joined together by amide bonds. The peptide sequence (AA¹)_{c} preferably is selected for enzyme-catalyzed cleavage by an enzyme in a location of interest in a biological system. For example, for conjugates that are targeted to but not internalized by a cell, a peptide is chosen that is cleaved by a protease that is in the extracellular matrix, e.g., a protease released by nearby dying cells or an amyloid-associated protease, e.g. membrane bound metalloproteases such as alpha secretase which cleaves APP at the cell surface, such that the peptide is cleaved extracellularly. For conjugates that are designed for internalization by a cell, the sequence (AA¹)_{c} preferably is selected for cleavage by an endosomal or lysosomal protease. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Preferably, (AA¹)_{c} contains an amino acid sequence (“cleavage recognition sequence”) that is a cleavage site by the protease. Many protease cleavage sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β-Ala-Leu-Ala-Leu (SEQ ID NO: 1). This can be combined with a stabilizing group to form succinyl-β-Ala-Leu-Ala-Leu (SEQ ID NO: 2). Other examples of suitable cleavable peptides are provided in the references cited below. Alternatively, linkers comprising a single amino acid residue can be used, as disclosed in WO 2008/103693.

In a preferred embodiment, the peptide sequence (AA¹)_{c} is chosen based on its ability to be cleaved by a lysosomal proteases, examples of which include cathepsins B, C, D, H, L and S. Preferably, the peptide sequence (AA¹)_{c} is capable of being cleaved by cathepsin B or serine protease neurosin, in vitro. Caspase cleavage of Tau protein. Also, serine protease neurosin (kallikrein-6) degrades α-synuclein and co-localizes with pathological inclusions such as Lewy bodies and glial cytoplasmic inclusions.

A growing amount of evidence indicates that MMPs may play an important role in the pathogenesis of Alzheimer's disease (AD). Peptide sequences designed to be cleaved by matrix metalloproteases (MMP)-2 and MMP-9 have been designed and tested for conjugates of dextran and methotrexate; PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, Pro-Val-Gly-Leu-11e-Gly (SEQ ID NO: 3), Gly-Pro-Leu-Gly-Val (SEQ ID NO: 4), Gly-Pro-Leu-Gly-11e-Ala-Gly-Gln (SEQ ID NO: 5), Pro-Leu-Gly-Leu (SEQ ID NO: 6), Gly-Pro-Leu-Gly-Met-Leu-Ser-Gln (SEQ ID NO: 7), and Gly-Pro-Leu-Gly-Leu-Trp-Ala-Gln (SEQ ID NO: 8). See, e.g., the previously cited references as well as Kline et al. (2004) Mol. Pharmaceut. 1:9-22 and Liu et al. (2000) Cancer Res. 60:6061-6067.

Yet another example is type II transmembrane serine proteases. This group of enzymes includes, for example, hepsin, testisin, and TMPRSS4. Gln-Ala-Arg is one substrate sequence that is useful with matriptase/MT-SP1 and Leu-Ser-Arg is useful with hepsin. (See, e.g., Lee et. al. (2000) J. Biol. Chem. 275:36720-36725 and Kurachi and Yamamoto, Handbook of Proeolytic Enzymes Vol. 2, 2^{nd} edition (Barrett A J, Rawlings N D & Woessner J F, eds) pp. 1699-1702 (2004)).

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Len-Ala-Leu, β-Ala-Leu-Ala-Leu (SEQ ID NO: 9), Gly-Phe-Leu-Gly (SEQ ID NO: 10), Val-Ala, Leu-Leu-Gly-Leu (SEQ ID NO: 11), Leu-Asn-Ala, and Lys-Leu-Val. Preferred peptides sequences are Val-Cit and Val-Lys.

In another embodiment, the amino acid located the closest to the drug moiety is selected from the group consisting of: Ala, Asn, Asp, Cit, Cys, Gln, Gln, Gly, Ile, Len, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Gln, Gly, Ile, Leu, Met, Phe, Pro, Ser, Thr, Trp, Tyr, and Val.

One of skill in the art can readily evaluate an array of peptide sequences to determine their utility in the present invention without resort to undue experimentation. See, for example, Zimmerman, M., et al., (1977) Analytical Biochemistry 78:47-51; Lee, D., et al., (1999) Bioorganic and Medicinal Chemistry Letters 9:1667-72; and Rano, T. A., et al., (1997) Chemistry and Biology 4:149-55.

A conjugate of this invention may optionally contain two or more linkers. These linkers may be the same or different. For example, a peptidyl linker may be used to connect the drug (i.e., cytoprotective agent) to the ligand (i.e., antibody) and a second peptidyl linker may attach a diagnostic agent (e.g., marker) to the antibody partner conjugate. Other uses for additional linkers include linking analytical agents, biomolecules, targeting agents, and detectable labels to the antibody-partner complex.

(v) Hydrazine Linkers (H)

In another embodiment, the conjugate of the invention comprises a hydrazine self-immolative linker, wherein the conjugate has the structure:

X⁴-(L⁴)ₚ-H-(L¹)ₘ-D

wherein D, L¹, L⁴, p, m, and X⁴ are as defined above and described further herein, and H is a linker comprising the structure:

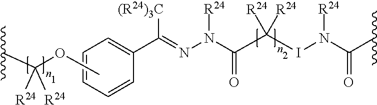

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

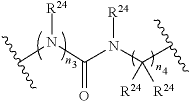

wherein n₃ is 0 or 1, with the proviso that when n₃ is 0, n₂ is not 0; and n₄ is 1, 2, or 3.

In one embodiment, the substitution on the phenyl ring is a para substitution. In certain preferred embodiments, n₁ is 2, 3, or 4 or n₁ is 3. In certain preferred embodiments, n₂ is 1. In certain preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Another hydrazine structure, H, has the formula:

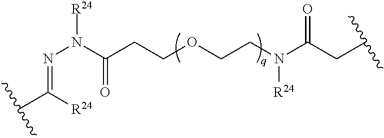

where q is 0, 1, 2, 3, 4, 5, or 6; and each R²⁴ is a member independently selected from the group consisting of H, substituted alkyl, unsubstituted alkyl, substituted heteroallyl, and unsubstituted heteroallyl. This hydrazine structure can also form five-, six-, or seven-membered rings and additional components can be added to form multiple rings.

The preparation, cleavage chemistry and cyclization kinetics of the various hydrazine linkers is disclosed in WO 2005/112919. As stated above, upon the hydrazine moiety is converted to a hydrazone when attached. This attachment can occur, for example, through a reaction with a ketone group on the L moiety. Therefore, the term “hydrazone linker” can also be used to describe the hydrazine linkers of the current invention.

(vi) Disulfide Linkers (J)

In yet another embodiment, the linker comprises an enzymatically cleavable disulfide group. In one embodiment, the invention provides a cytoprotective antibody-partner compound having a structure according to Formula (d):

X⁴L⁴ₚJL¹ₘD

wherein D, L¹, L⁴, p, m, and X⁴ are as defined above and described further herein, and J is a disulfide linker comprising a group having the structure:

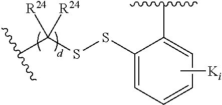

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0, 1, 2, 3, or 4; and d is an integer of 0, 1, 2, 3, 4, 5, or 6.

The aromatic ring of a disulfide linker can be substituted with one or more “K” groups. A “K” group is a substituent that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents include, but are not limited to, F, Cl, Br, I, NO₂, OH, OCH₃, NHCOCH₃, N(CH₃)₂, NHCOCF₃ and methyl. For “Ki”, i is an integer of 0, 1, 2, 3, or 4. In a specific embodiment, i is 0.

In a preferred embodiment, the linker comprises an enzymatically cleavable disulfide group of the following formula:

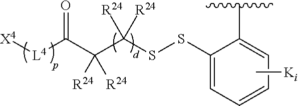

wherein L⁴, X⁴, p, and R²⁴ are as described above, and d is 0, 1, 2, 3, 4, 5, or 6. In a particular embodiment, d is 1 or 2.

A more specific disulfide linker is shown in the formula below:

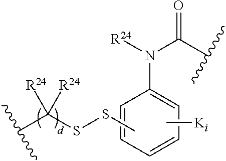

Preferably, d is 1 or 2 and each K is H.

Another disulfide linker is shown in the formula below:

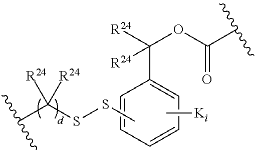

Preferably, d is 1 or 2 and each K is H.

In various embodiments, the disulfides are functionalized at the ortho position. In another specific embodiment, d is 0. In certain preferred embodiments, R²⁴ is independently selected from H and CH₃.

The preparation and use of disulfide linkers such as those described above is disclosed in WO 2005/112919.

In a specific embodiment, a linker of the invention is selected from the group consisting of peptide-based linkers such as citrulline-valine linkers, (derived from maleimidocaproyl-valinecitrulline-p-aminobenzyloxycarbonyl), and linkers derived from lysine residues, disulfide linkers derived from N-succinimydyl 4-(2-pyridyldithio)-pentanoate (SPP), and thioether linkers derived from succinimidyl-4-(N-maleimidomethyl)cyclohexane-1-carboxylate (MCC), bis-maleimido-trioxyethylene glycol [see, Junutula et al. (2010) Clin Cancer Res; 16(19); 4769-78];, or maleimidocaproyl.

For further discussion of types of linkers and the conjugation of therapeutic agents to antibodies, see also U.S. Pat. No. 7,087,600; U.S. Pat. No. 6,989,452; U.S. Pat. No. 7,129,261; US 2006/0004081; US 2006/0247295; WO 02/096910; WO 2007/051081; WO 2005/112919; WO 2007/059404; WO 2008/083312; WO 2008/103693; Saito et al. (2003) Adv. Drug Deliv. Rev. 55:199-215; Trail et al. (2003) Cancer Immunol. Immunother. 52:328-337; Payne. (2003) Cancer Cell 3:207-212; Allen (2002) Nat. Rev. Cancer 2:750-763; Pastan and Kreitman (2002) Curr. Opin. Investig. Drugs 3:1089-1091; Senter and Springer (2001) Adv. Drug Deliv. Rev. 53:247-264.

### (IV.) Methods of Conjugation

Partner molecules and antibodies of the invention may be conjugated using any suitable technique known in the art. For example, the Mannich reaction offers the possibility of conjugating melatonin directly to solvent accessible lysine residues. The Mannich reaction consists of the condensation of formaldehyde (or sometimes another aldehyde) with ammonia, in the form of its salt, and another compound containing an active hydrogen. See, Vollhardt and Schore, Organic Chemistry: Structure and Function, Fourth Edition, W. H. Freeman & Co. © 2002 W. H. Freeman & Co., and Sumanas, Inc. Instead of using ammonia, however, this reaction can also be done with primary or secondary amines, or even with amides. An example of this reaction is illustrated in the condensation of acetophenone, formaldehyde, and a secondary amine salt: C6H5COCH3+CH2O+R2NH.HCl—C6H5COCH2CH2NR2.HCl+H2O. Melatonin was previously conjugated to bovine serum albumin using this method, and can involve cross-linking at position 1 of the indole ring to the free amine of lysine residues in the protein. See, Amines and their metabolites (1985) by Alan A. Boulton, Glen B. Baker, Judith M. Baker, page 271.

The skilled artisan will understand that the specific method to be used for conjugation of antibody to partner molecule will depend up on the specific linker and partner molecule being used. By way of example, and without limitation, provided herein is a description of conjugation of an antibody to a drug using Succinimidyl-4-(N-maleimidomethyl)-cyclohexane-1-carboxylate (SMCC, Pierce, Rockford, Ill.). SMCC is dissolved in dimethylacetamide (DMA) and added to the antibody solution to make a final SMCC/Ab molar ratio of 10:1. The reaction is allowed to proceed for 3 hours at room temperature with mixing. The SMCC-modified antibody is subsequently purified on a GE Healthcare HiTrap desalting column (G-25) equilibrated in 35 mM sodium citrate with 150 mM NaCl and 2 mM EDTA, pH 6.0. The drug (e.g. melatonin) is dissolved in DMA, and added to the SMCC-antibody preparation to give a molar ratio of melatonin to antibody of 10:1. The reaction is allowed to proceed for 4 to 20 hours at room temperature with mixing. The melatonin-modified antibody solution is diafiltered with 20 volumes of phosphate-buffered saline to remove unreacted melatonin, sterile-filtered, and stored at 4° C. Typically, a 40% to 60% yield of antibody is achieved through this process. The preparation is usually greater than 95% monomeric as may be assessed by gel filtration and laser light scattering. Typically, the drug to antibody ratio is expected to be between about 2.5 and 4.5. See, Polson et al. (2007) Blood 110(2)616-623.

### (IV.) Pharmaceutical Compositions

While compositions of the invention may be administered alone, it may be preferable in some embodiments to administer them as a pharmaceutical formulation.

Pharmaceutical compositions include an active agent and a pharmaceutically acceptable carrier, excipient, or diluent. Pharmaceutically acceptable carriers, including diluents or excipients, for therapeutic use are well known in the pharmaceutical art, and are described herein and, for example, in Remington's Pharmaceutical Sciences, Mack Publishing Co. (A. R. Gennaro, ed., 18^{th} Edition (1990)) and in CRC Handbook of Food, Drug, and Cosmetic Excipients, CRC Press LLC (S. C. Smolinski, ed. (1992)). The term “carrier” applied to pharmaceutical compositions of the invention refers to a diluent, excipient, or vehicle with which a compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E.W. Martin, 18th Edition.

For human therapy, the pharmaceutical compositions, including each of the active agents, will be prepared in accordance with good manufacturing process (GMP) standards, as set by the Food & Drug Administration (FDA). Quality assurance (QA) and quality control (QC) standards will include testing for purity and function and other standard measures.

A preferred delivery vehicle is any chemical entity that ensures delivery of an inhibitor to the target site (e.g., brain, or more specifically, an amyloid deposit or neurofibrillary tangle) in a selective manner, achieves sufficient concentration of active inhibitor at the target site, and is preferably bioavailable in the brain.

In certain embodiments, it is preferred that the compositions of the invention be able to cross the blood brain barrier (BBB), in order to facilitate treatment of neuropathogenic diseases and disorders. The usefulness of IV Ig treatment in epilepsy was assessed by Engelen B G et al. (J. Neurol Neurosurg Psychiatry 1994 November 57 Supp 21-5). The conclusion of these authors from the study on cerebrospinal fluid IgG concentrations before and after IV Ig treatment in patients with epilepsy was that the main component of IV Ig preparation crosses the blood CSF barrier and significantly increases CSF IgG concentration, and may reach the brain and act centrally. The presence of IgG in the CNS was demonstrated by immunocytochemistry and showed a close anatomical relationship between the distribution of this protein and the blood-brain barrier. IgG was immunolocalized in the normal rat brain by using monoclonal and polyclonal antibodies to IgG and its subclasses. Further, the passage of intravenous immunogloubulin and interactions with the CNS was summarized in a review by Wurster et al. (J. Neurol Neurosurg Psychiatry 1994 November 57 Supp 21-5).

In certain embodiments, the ADC of the invention penetrate into the brain cells, through the blood brain barrier (BBB), by using methods or carriers, which details are provided below. For example, and without limitation, preferred compounds that may be added to formulations to enhance the solubility of the ADC of the present invention are cyclodextrin derivatives, preferably hydroxypropyl-gamma-cyclodextrin. Drug delivery vehicles containing a cyclodextrin derivative for delivery of peptides to the central nervous system are described in Bodor, N., et al. (1992) Science 257:1698-1700.

Accordingly, use of an ADC of the invention in combination with a cyclodextrin derivative may result in greater inhibition of 13 amyloid neurotoxicity than use of the ADC alone. Chemical modifications of cyclodextrins are known in the art (Hanessian, S., et al. (1995) J. Org. Chem. 60:4786-4797). In addition to use as an additive in an ADC-containing composition (including pharmaceutical composition) of the invention, cyclodextrin derivatives may also be useful as modifying groups and, accordingly, may also be covalently coupled to antibodies comprised in ADC of the invention.

Low et al., U.S. Pat. No. 5,108,921, reviews available methods for transmembrane delivery of molecules such as proteins and nucleic acids by the mechanism of receptor mediated endocytotic activity. These receptor systems include those recognizing galactose, mannose, mannose-6-phosphate, transferrin, asialoglycoprotein, transcobalamin (vitamin B.sub.12), α-2 macroglobulins, insulin and other peptide growth factors such epidermal growth factor (EGF). Low et al. also teaches that nutrient receptors, such as receptors for biotin and folate, can be advantageously used to enhance transport across the cell membrane due to the location and multiplicity of biotin and folate receptors on the membrane surfaces of most cells, and the associated receptor mediated transmembrane transport processes. Thus, a complex formed between a compound to be delivered into the cytoplasm and a ligand, such as biotin or folate, is contacted with a cell membrane bearing biotin or folate receptors to initiate the receptor mediated trans-membrane transport mechanism and thereby permit entry of the desired compound into the cell.

A biotin ligated can be attached to a DM molecule, for example, by incorporating commercially available biotinylated deoxynucleotide triphosphates, e.g., biotin-14-MTP or biotin-14dCTP from Invitrogen Life Technologies, Carlsbad, Calif., using terminal deoxynucleotidyl transferase (Karger, B. D., 1989). Biotin-14dATP is a MTP analog with biotin attached at the 6-position of the purine base by a 14-atom linker and biotin-14-dCTP is a dCTP analog with biotin attached at the N.sup.4-position of the pyrimidine base also by a 14-atom linker.

In one embodiment, the ADC of the invention can be delivered by liposomes, as discussed, supra.

In another approach for enhancing transport across the BBB, and ADC of the invention is conjugated to a second peptide or protein thereby forming a chimeric protein, wherein the second peptide or protein undergoes absorptive-mediated or receptor-mediated transcytosis through the BBB. Accordingly, by coupling the ADC to this second peptide or protein, the chimeric protein is transported across the BBB. The second peptide or protein can be a ligand for a brain capillary endothelial cell receptor ligand. For example, a preferred ligand is a monoclonal antibody that specifically binds to the transferrin receptor on brain capillary endothelial cells (see e.g., U.S. Pat. Nos. 5,182,107 and 5,154,924 and PCT Publications WO 93/10819 and WO 95/02421, all by Friden et al.). Other, suitable peptides or proteins that can mediate transport across the BBB include histones (see e.g., U.S. Pat. No. 4,902,505 by Pardridge and Schimmel) and ligands such as biotin, folete, niacin, pantothenic acid, riboflavin, thiamin, pryridoxal and ascorbic acid (see e.g., U.S. Pat. Nos. 5,416,016 and 5,108,921, both by Heinstein). Additionally, the glucose transporter GLUT-1 has been reported to transport glycopeptides (L-serinyl-β-D-glucoside analogues of [Met5]enkephalin) across the BBB (Polt, R. et al. (1994) Proc. Natl. Acad. Sci. USA 91:7114-1778). Accordingly, an ADC described herein can be coupled to such a glycopeptide to target the ADC to the GLUT-1 glucose transporter. For example, an ADC comprising an antibody which is modified at its amino terminus with the modifying group Aic (3-(O-aminoethyl-iso)-cholyl, a derivative of cholic acid having a free amino group) can be coupled to a glycopeptide through the amino group of Aic by standard methods. Chimeric proteins can be formed by recombinant DNA methods (e.g., by formation of a chimeric gene encoding a fusion protein) or by chemical crosslinking of the antibody of the ADC to the second peptide or protein to form a chimeric protein. Numerous chemical crossing agents are known (e.g., commercially available from Pierce, Rockford Ill.). A crosslinking agent can be chosen which allows for high yield coupling of the antibody to the second peptide or protein and for subsequent cleavage of the linker to release bioactive ADC. For example, a biotin-avidin-based linker system may be used.

In yet another embodiment for enhancing transport across the BBB, the ADC is encapsulated in a carrier vector, which mediates transport across the BBB. For example, the ADC can be encapsulated in a liposome, such as a positively charged unilamellar liposome (see e.g., PCT Publications WO 88/07851 and WO 88/07852, both by Faden) or in polymeric microspheres (see e.g., U.S. Pat. No. 5,413,797 by Khan et al., U.S. Pat. No. 5,271,961 by Mathiowitz et al. and U.S. Pat. No. 5,019,400 by Gombotz et al.). Moreover, the carrier vector can be modified to target it for transport across the BBB. For example, the carrier vector (e.g., liposome) can be covalently modified with a molecule which is actively transported across the BBB or with a ligand for brain endothelial cell receptors, such as a monoclonal antibody that specifically binds to transferrin receptors (see e.g., PCT Publications WO 91/04014 by Collins et al. and WO 94/02178 by Greig et al.).

An ADC of the invention can be formulated into a pharmaceutical composition wherein the ADC is the only active compound or, alternatively, the pharmaceutical composition can contain additional active compounds. For example, two or more active agents may be used in combination (e.g. two or more ADC, or an ADC in combination with another active agent). Moreover, an ADC of the invention can be combined with one or more other agents that have anti-amyloidogenic and/or anti-fibrillogenic properties. For example, an ADC can be combined with the non-specific cholinesterase inhibitor tacrine (COGNEX™, Parke-Davis).

### (VI.) Methods of Treatment

(A) Methods for Treating Proteinopathies

As described herein, it is presently discovered that antibodies specific for amyloid polypeptides (e.g., Aβ) and amyloid-associated polypeptides (e.g., tau or APP) can be used to target cytoprotective agents such as antioxidants, to a site of damage in proteinopathies (e.g., an amyloid deposit). While not intending to be bound by theory or a particular mechanism of action, the ADC can therefore provide the dual benefit of the protein-clearing action of the antibody, coupled to specific targeting of a cytoprotective agent to a site in need of such cytoprotection. Thus, the compositions of the invention are useful both for clearing amyloid deposits and for providing cytoprotective (e.g., neuroprotective) effects to the targeted cell. Moreover, in a specific embodiment, the cytoprotective agent is melatonin, which not only provides beneficial antioxidant effects, but can also interact with the high affinity copper binding site in Aβ and inhibit fibril formation.

In certain aspects, the invention thus provides methods for inhibiting accumulation of amyloid polypeptides or amyloid-associated polypeptides in the brain of a patient suffering from a proteinopathy. In certain embodiments, the method comprises contacting in vivo soluble amyloid polypeptides, amyloid-associated polypeptides, tau, or other polypeptide involved in a proteinopathy (i.e., target polypeptide) in said patient with an ADC composition of the invention. The site of contact of the ADC and the target polypeptide will vary depending on the route of delivery and the specific proteinopathy being treated. In one embodiment, the site of contact is in the cerebrospinal fluid of the patient. In other embodiments, such as, e.g., for treating a peripheral amyloidosis (e.g., type II diabetes or serum amyloidosis), the contact site will be a peripheral site (non CNS), such as, but not limited to, in the blood, liver, spleen, kidney, adrenal, heart, or pancreas or other organ.

In another embodiment, the invention provides a method for treating or delaying onset of a proteinopathy, comprising administering to a subject in need thereof an effective amount of an ADC composition of the invention, to inhibit the formation of fibrils or the formation of amyloid or amyloid-like deposits associated with amyloidosis-related diseases, or to inhibit the formation of neurofibrillary tangles associated with tauopathies.

In another embodiment, the invention provides a method for promoting clearance of aggregates from the brain of a subject, comprising administering to the subject in need thereof an effective amount of an ADC composition of the invention, wherein said polypeptide is tau, under conditions and in an amount effective to promote clearance of neurofibrillary tangles from the brain of the subject.

Proteinopathies that can be treated according to the present invention include, but are not limited to: (1) neuropathic conditions associated with Aβ amyloidosis, such as age related macular degeneration (Aβ), glaucoma, traumatic brain injury, cerebral amyloid angiopathy (CAA), hereditary cerebral hemorrhage with amyloidosis Dutch type (HCHWA-D) AD, early onset familial AD (EOFAD), and Down Syndrome; as well as (with the amyloidogenic protein involved in the disease or disorder is shown in parentheses): Parkinson's disease (PD) (α-synuclein), Huntington's disease (Huntingtin protein), amyotrophic lateral sclerosis (superoxide dismutase), Pick's complex (Tau), familial frontotemporal dementias (Tau) and prion disease (pathogenic prion, PrPSc); and (2) non-neuropathic amyloid-associated diseases and disorders, such as, but not limited to, diabetes mellitus type 2 (IAPP (Amylin)) (the amyloidogenic protein involved in the disease or disorder is shown in parentheses), medullary carcinoma of the thyroid (calcitonin), isolated atrial amyloidosis (atrial natriuretic factor (AANF)), atherosclerosis (apolipoprotein AI (AApoA1)), rheumatoid arthritis (serum amyloid A (AA)), aortic medial amyloid (medin (AMed)), familial amyloid polyneuropathy (transthyretin (ATTR)), hereditary non-neuropathic systemic amyloidosis (lysozyme (ALys)), dialysis related amyloidosis (beta 2 microglobulin (Aβ2M), Finnish amyloidosis (Gelsolin (AGel)), Lattice corneal dystrophy (keratoepithelin (AKer)), cerebral amyloid angiopathy (beta amyloid (Aβ)), cerebral amyloid angiopathy (Icelandic type) (cystatin (ACys)), systemic AL amyloidosis (Immunoglobulin light chain AL (AL)), and sporadic inclusion body myositis (amyloid precursor protein, beta amyloid, presenilin1, sequestosome1 (p62), TAR DNA binding protein-43 (TDP-43), ubiquitinated-proteins, apolipoprotein E, alpha-synuclein and phosphorylated tau).

In certain embodiments, once delivered into the brain an ADC will transfer into the extracellular space, interstitial fluid and cerebrospinal fluid. The specific antibodies and/or cytoprotective agents (e.g., melatonin) of the ADC then form a soluble complex with the target polypeptide (e.g., Aβ peptide, tau, prion PrPSc). These soluble complexes reduce, in one embodiment, the deposition of Aβ peptides into amyloid plaques and attenuate Aβ peptide-induced neurotoxicity by clearing Aβ peptides from the central nervous system through drainage of the extracellular space, interstitial fluid and cerebrospinal fluid into the general blood circulation where they will be eliminated by protease digestion. The cytoprotective agent in the conjugate provides (e.g., melatonin) provides additional protective effects (e.g., antioxidant effects). Accordingly, the accumulation of newly secreted soluble Aβ peptides responsible for amyloid deposition and Aβ-induced neurotoxicity is inhibited and cytoprotection is conferred. ADCs can also bind directly to insoluble plaque causing clearance through Fc-mediated phagocytosis by microglia. In some cases ADCs may be internalized and bind intracellular amyloid.

Furthermore, clearance of amyloid-β peptides in accordance with the present invention is expected to reduce the inflammatory process observed in Alzheimer's disease and other amyloidogenic diseases or disorders by inhibiting, for example, amyloid β-induced complement activation and cytokine release, and blocking also the interaction of Aβ with cell surface receptors such as the RAGE receptor.

In another embodiment, once an ADC of the invention binds to the amyloid β peptide the ADC can elicit a cellular immune response (e.g., via activation of the Fc receptor). Fc receptor can distinguish between an antibody, which is bound to an antigen, and a free antibody. The result will be that the Fc receptors will enable accessory cells, which are usually not capable of identifying target antigens to target and engulf Aβ thereby eliminating the requirement for a stoichiometric relationship between antibody and antigen. As a consequence, less ADC will be required to penetrate the BBB for elimination of deposited amyloid β peptides.

In another embodiment, the interaction of amyloid β with APOE4 gene product will be reduced following administration of an ADC of the invention.

In another embodiment, the pharmaceutical composition and the antibodies of the present invention will delay the onset and inhibit or suppress the progression of a proteinopathy by having a peripheral effect. The clearance or the removal of amyloid beta from the periphery will change the equilibrium of the amyloid beta in the blood and as a result in the brain. Recent studies have shown that amyloid beta is transported from the cerebrospinal fluid to the plasma with an elimination half-life from brain of about half an hour. Thus, the ADC can affect the amyloid beta level in the plasma, cause accumulation of central amyloid beta in the plasma and as a result reduce the amyloid β deposition in the brain.

(B) Proteinopathy Assays

A number of biological assays are available to evaluate and to optimize the ADC compositions of the invention in vitro and in vivo.

For example, to assess the cytoprotective effect of ADC of the invention in vitro, cells (e.g., PC12 cells ATCC (Catalog #CRL-1721)) exposed to Aβ (23-35) or the superoxide dismutase (SOD) inhibitor diethyldithiocarbamic acid (DDTC), which are oxidotoxins, may be treated with an ADC of the invention (or, in parallel, with melatonin or antibody alone, or with media alone) and the degree of lipid peroxidation can be measured. Under these experimental conditions, the degree of lipid peroxidation can be estimated by measuring the formation of malondialdehyde acid (MDA) in cell lysates as described. See, Omar, R. A., et al. (1987) Cancer Res 47:3473-3476.

Binding affinity of the ADC may be measured according to any suitable technique known in the art. The following methods are described as a non-limiting example. Affinity measurements of an ADC may be carried out using BiaCore kinetic analysis (Surface Plasmon Resonance (SPR)). For example, the ability of an ADC to immobilize high levels (high density) or low density of soluble Aβ1-40 can be measured using a Biacore T100 using CMS sensor chips (Cat: BR-1005-30). For measuring high density Aβ1-40 binding, soluble and aggregated Aβ1-40 (Bachem H-1194 and H-5568, respectively) is immobilized onto a sensor chip coated with carboxymethylated dextran (Biacore CM5). A BSA coated flow cell may be used as a blank Amine coupling may be performed as follows: activation is carried out through the injection of a 1:1 EDC:NHS mixture for 7 min then coupling performed by injecting ligand at 20 μg/mL diluted in 100 mM sodium acetate (PH 3.8) at 10 μL/min for 7 minutes. The Aβ peptides are immobilized until saturation. Similarly BSA (Sigma A 7030) at 30 μg/mL is injected at 10 μL/min for 7 minutes. The remaining activated carboxyl groups were blocked with 1 M ethanolamine (pH 8.5). The ADC are diluted in running buffer HBS-N in concentrations ranging from 1000 nM (150 μg/mL) to 31.25 nM and then injected at a flow rate of 20 μL/min for 3 min. Buffer is allowed to flow over the surface for 5 min for dissociation data. Regeneration of the flow cells is regenerated by a single 30 s pulse with 100 mM HCl at 10 μL/min. The kinetics of binding/dissociation is analyzed according to the 1:1 interaction model using BIAcore T100 evaluation software package 2.0.

Low density immobilization of soluble and aggregated Aβ1-40 may be assayed as follows: soluble Aβ1-40 is immobilized as described above with the following modifications. Aβ1-40 is diluted to a final concentration of 0.25 μg/mL and coupled for 50 s at 10 μL/min. Flow cell I is treated to the coupling reaction but in the absence of peptide. Aggregated Aβ1-40 is treated in the same manner with flow cells 3 used as the blank control and peptide coupled to flow cell 4. A final response level is determined.

The SPR conditions for affinity determination are set as follows: ADC binding to the soluble peptide Aβ1-40 are treated as follows: the mAbs are diluted in running buffer HBS-EP (GE Healthcare cat: BR-1OOI-88) plus 1 mg/ml of carboxymethyl dextran (GE Healthcare BR-1006-91) at concentrations ranging from 80 nM (12 μg/mL) to 1.25 nM and then injected at a flow rate of 60 μL/min for 160 sec. Buffer is allowed to flow over the surface for 10 min for dissociation data. Regeneration of the flow cells is carried out by a single 30 s pulse with 100 mM HCl at 10μL/min. The kinetics of binding/dissociation can be analyzed according to the 1:1 interaction model using BIAcore T100 evaluation software package 2.0. ADC binding to the aggregated peptide Aβ1-40 may be treated as follow:

The ADC are diluted in running buffer HBS-EP plus μg/ml of carboxymethyl dextran at concentrations ranging from 160 nM (24 μg/mL) to 1.25 nM and then injected at a flow rate of 60 μL/min for 100 sec. Buffer is allowed to flow over the surface for 15 min for dissociation data. Regeneration of the flow cells is regenerated by a single 30 s pulse with 150 mM HCl at 10 μL/min. The kinetics of binding/dissociation is analyzed according to the 1:1 interaction model using BIAcore T100 evaluation software package 2.0.

Other methods for assaying activity of ADC of the invention are described, e.g., in Pappolla et al., supra, and Cheng et al., supra.

In certain embodiments, it may be desirable to determine the immunogenicity of an ADC of the invention. Preferably, the ADC have low immunogenicity. Assays for determining whether an ADC is immunogenic are described, e.g., in Van Walle I, et al. (2007) Expert Opin Biol Ther.; 7:405-18. Furthermore, commercial services for assaying immunogenicity are available, e.g., from Lonza (Basel, Switzerland).

For characterizing the properties of an ADC of the invention in vivo, deposition of amyloid β(1-40) and amyloid β(1-42) or other amyloidogenic protein may be determined in animal models following treatment with an ADC composition of the invention. For such assays, suitable mammalian (e.g., murine) models may be used, such as, e.g., Tg2576 mice genetically engineered to express amyloid precursor proteins (APP) Hsiao, K, et al. (1996) Science 274:99-102; or APP/PS1 Holcomb L., et al. (1998) Nature. Med, 4: 97-100; mice expressing mutant (P301L) tau protein Lewis et al. (2000) Nature Genetics 25:402-405; PD models, see Harvey B K, et al. Acta Neurochir Suppl. 2008; 101:89-92; for HD transgenic mice see P. Hemachandra et al. (1998) Genetics and Molecular Nature Genetics 20:198-202; ALS mice are described, e.g., at www.researchals.org/.../p41_jax_sod1manual_{—}20091202_{—}29aPcx.pdf. In such models, mice, e.g., transgenic mice, that are predisposed to develop a proteinopathy, such as AD, PD, HD, etc., are injected with an ADC of the invention, or a control (e.g., melatonin alone, antibody alone, or PBS). Following the treatment course, which will depend upon the specific animal model, symptoms of the proteinopathy, e.g., appearance of amyloid deposits are assessed, using, e.g., immunohistochemistry or other imaging techniques known in the art. ELISA assays can also be used to quantify levels of amyloid β e.g., in brain homogenate samples. See, Arendash et al. (2001) DNA and Cell Biology 20(11)737-744. Other suitable animal models of proteinopathies are known in the art, and are also included. The above-described animal models are provided as examples and are not limiting.

Preferably, an ADC composition of the invention reduces amyloid β deposition by at least 10%, at least 15%, at least 20%, at least 25%, at least 30%, at least 35%, at least 40%, at least 45%, at least 50%, at least 55%, at least 60%, at least 65%, at least 75%, or more.

(C) In Vivo Imaging

In certain embodiments ADC of the invention are useful for in vivo imaging. In particular, an ADC comprising a labeled partner molecule (e.g., labeled antioxidant molecule such as melatonin labeled with, e.g., a radiolabel, a fluorescent label (e.g., GFP), or an enzyme that catalyzes a detectable modification to a substrate (e.g., horseradish peroxidase)) may be administered to a subject for diagnostic purposes. For example, it may be desirable to determine whether a subject (e.g., a subject with a suspected AD or PD diagnosis) has detectable amyloid or amyloid-like deposits in the brain (or other site, e.g., pancreas, kidneys, liver, etc.). An ADC comprising an antibody targeted to a polypeptide found in the amyloid deposit (e.g., Aβ or other amyloid-associated protein) and containing a detectable marker may be administered to the subject, and imaging can be carried out according to suitable methods known in the art.

Imaging in human brains using positron emission tomography (PET) is described in detail, for example, in Wagner et al. (1983) Science 221(4617):1264-1266. Wagner describes intravenous injection of ¹⁸F or ¹¹C labeled compounds, which are then detected in vivo by PET. Such radiolabels and other suitable markers are contemplated for use herein. In animal models, ADC of the invention may be used for diagnostic imaging by MRI or PET or other suitable method of detection known in the art.

While it is possible that the antibody in the ADC can be directly labeled with a marker, in certain embodiments, it is preferred that the partner molecule is labeled with the marker. In certain embodiments, the partner molecule is a small molecule that can be more easily labeled compared to the antibody.

In a particularly preferred embodiment, the cytoprotective partner molecule (e.g., melatonin or indole-3-propionic acid, etc.) is directly synthesized with a marker (e.g., radiolabel, such as ¹⁸F, ¹¹C or ¹⁴C). Direct synthesis of the cytoprotective agent to contain a detectable marker advantageously avoids the need for an additional conjugation step when preparing the ADC of the invention. Of course, it is to be understood that certain markers are preferably conjugated to the partner molecule after synthesis of the partner molecule.

### (VII.) Administration and Dosage

ADC-containing compositions of the invention can be subcutaneously, intravenously, intradermally, intramuscularly, intaperitoneally, intracerebrally, intranasally, orally, transdermally, buccally, intra-arterially, intracranially, or intracephalically. A particularly preferred route of administration of an ADC is intravenous.

An individual in need thereof is, for example, a human or other mammal that would benefit by the administration of an ADC described herein, such as a human or other mammal suffering from or at risk of developing a proteinopathy as described herein (e.g., AD, PD, HD, ALS, prion disease, etc.), as determined, e.g., by the individual's physician.

According to this disclosure, an ADC-containing composition (including pharmaceutical compositions) of this disclosure can be introduced parenterally, transmucosally, e.g., orally (per os), nasally, or rectally, or transdermally. Parental routes include intravenous, intra-arteriole, intra-muscular, intradermal, subcutaneous, intraperitoneal, intraventricular, and intracranial administration. Specific organs may be targeted, e.g., brain, by direct administration to the targeted organ.

For oral administration (e.g., buccal), the pharmaceutical compositions may take the form of tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the chaperones for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The pharmaceutical compositions may be added to a retained physiological fluid such as blood or synovial fluid.

In another embodiment, the active ingredient can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

ADC-containing compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the formulations described previously, ADC-containing compounds and compositions may also be formulated as a depot preparation. Such long acting formulations may be administered by implantation (for example subcutaneously or intramuscularly) or by intramuscular injection. Thus, for example, the compounds and compositions may be formulated with suitable polymeric or hydrophobic materials (for example, as an emulsion in an acceptable oil) or ion exchange resins, or as sparingly soluble derivatives, for example, as a sparingly soluble salt. In yet another embodiment, the therapeutic compound can be delivered in a controlled release system. For example, an ADC-containing composition may be administered using intravenous infusion with a continuous pump, in a polymer matrix such as poly-lactic/glutamic acid (PLGA), a pellet containing a mixture of cholesterol and the active ingredient (Silastic®; Dow Corning, Midland, Mich.; see U.S. Pat. No. 5,554,601) implanted subcutaneously, an implantable osmotic pump, a transdermal patch, liposomes, or other modes of administration.

Administration of ADC-containing compositions may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

For in vivo prevention of cytotoxic effects, a preferred dosage of melatonin, when administered by itself, is between about 1 μg and about 100 g of melatonin. Desirable serum concentrations of melatonin are in the range of about 50 μM to about 100 μM. The actual preferred amount of melatonin to be administered according to the present invention will vary according to the particular form of melatonin (for example, melatonin or an analog thereof), the particular composition formulated, and the mode of administration. Specifically, ADC of the invention advantageously achieve high localized concentration of melatonin or other cytoprotective agent. Thus, in preferred embodiments, the dosage of melatonin is between about 0.01 ng and 1 μg. A preferred concentration of antibody in the ADC conjugate ranges from about 0.05 mg/kg to 10 mg/kg.

Many factors that may modify the action of a composition of the invention can be taken into account by those skilled in the art; e.g., body weight, sex, diet, time of administration, route of administration, rate of excretion, condition of the subject, drug combinations, and reaction sensitivities and severities. Administration can be carried out continuously or periodically within the maximum tolerated dose. Optimal administration rates for a given set of conditions can be ascertained by those skilled in the art using conventional dosage administration tests.

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

Reagents and solvents used below can be obtained by commercial sources such as Aldrich Chemical Co. (Milwaukee, Wis., USA). Mass spectrometry results are reported as the ratio of mass over charge for the M+H ion containing the most common atomic isotopes.

### Example 1

### (2R)-2-amino-3-((1-(6-((2-(5-methoxy-1H-indol-3-yl)ethyl)amino)-6-oxohexyl)-2,5-dioxopyrrolidin-3-yl)thio)propanoic acid

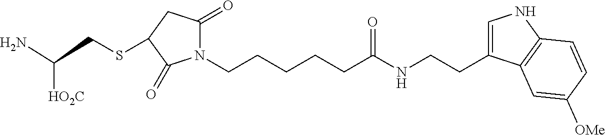 [SMILES: COc1ccc2[nH]cc(CCNC(=O)CCCCCN3C(=O)CC(SC[C@H](N)C(=O)O)C3=O)c2c1]

### Step 1. Preparation of 6-(2,5-dioxo-2,5-dihydro-1H-pyrrol-1-yl)-N-(2-(5-methoxy-1H-indol-3-yl)ethyl)hexanamide)

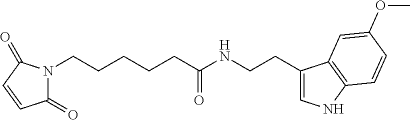 [SMILES: COc1ccc2[nH]cc(CCNC(=O)CCCCCN3C(=O)C=CC3=O)c2c1]

To a solution of 2,5-dioxopyrrolidin-1-yl 6-(2,5-dioxo-2,5-dihydro-1H-pyrrol-1-yl)hexanoate (247 mg, 0.81 mmol) in dichloromethane (10 mL) was added 2-(5-methoxy-1H-indol-3-yl)ethanamine (152 mg, 0.81 mmol) and the mixture was stirred at rt for minutes. The succinimide precipitated as a white solid and LCMS analysis indicated an 89% yield of the desired product and an 11% yield of the side product formed by addition of the amine to the double bond. The reaction mixture was filtered and concentrated in vacuo to afford 6-(2,5-dioxo-2,5-dihydro-1H-pyrrol-1-yl)-N-(2-(5-methoxy-1H-indol-3-yl)ethyl)hexanamide) (300 mg, isolated yield 86%, purity 88%) as a yellow oil. MS 384.2 (M+H⁺).

### Step 2. Preparation of (2R)-2-amino-3-((1-(6-((2-(5-methoxy-1H-indol-3-yl)ethyl)amino)-6-oxohexyl)-2,5-dioxopyrrolidin-3-yl)thio)propanoic acid

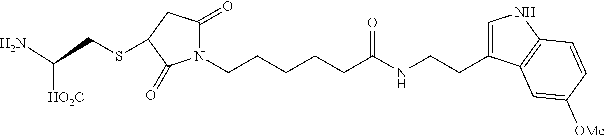 [SMILES: COc1ccc2[nH]cc(CCNC(=O)CCCCCN3C(=O)CC(SC[C@H](N)C(=O)O)C3=O)c2c1]

6-(2,5-dioxo-2,5-dihydro-1H-pyrrol-1-yl)-N-(2-(5-methoxy-1H-indol-3-yl)ethyl)hexanamide) (307 mg, 0.801 mmol) was dissolved in N,N-dimethylformamide (dry) (10 mL). L-cystein (388 mg, 3 2 mmol) was added and the mixture was stirred at rt overnight. The mixture was concentrated in vacuo to afford the crude product as a white solid. Purification by flash column chromatography (5-100% MeCN in H₂O) and subsequent lyophilization afforded (2R)-2-amino-3-((1-(6-((2-(5-methoxy-1H-indol-3-yl)ethyl)amino)-6-oxohexyl)-2,5-dioxopyrrolidin-3-yl)thio)propanoic acid as a white solid (100 mg, isolated yield 24.75%). MS 505.2 (M+H⁺).

### Example 2

### (2R)-2-amino-3-((1-(1-(5-methoxy-1H-indol-3-yl)-4,20-dioxo-7,10,13,16-tetraoxa-3,19-diazadocosan-22-yl)-2,5-dioxopyrrolidin-3-yl)thio)propanoic acid

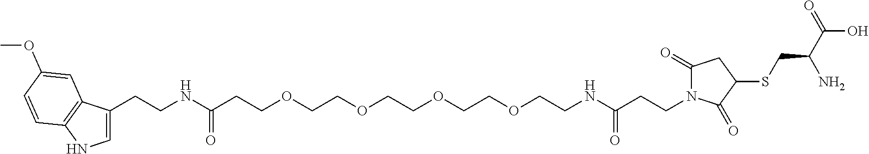 [SMILES: COc1ccc2[nH]cc(CCNC(=O)CCOCCOCCOCCOCCNC(=O)CCN3C(=O)CC(SC[C@H](N)C(=O)O)C3=O)c2c1]

### Step 1. Preparation of 1-(3-(2,5-dioxo-2,5-dihydro-1H-pyrrol-1-yl)propanamido)-N-(2-(5-methoxy-1H-indol-3-yl)ethyl)-3,6,9,12-tetraoxapentadecan-15-amide

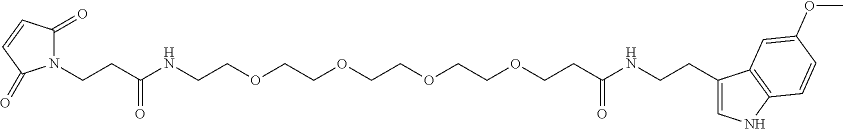 [SMILES: COc1ccc2[nH]cc(CCNC(=O)CCOCCOCCOCCOCCNC(=O)CCN3C(=O)C=CC3=O)c2c1]

2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate (200 mg, 0.389 mmol) and 2-(5-methoxy-1H-indol-3-yl)ethanamine (70.4 mg, 0.370 mmol) were dissolved in N,N-dimethylformamide (2 mL) and stirred at rt for 30 min. The reaction was then filtered and concentrated in vacuo to afford 1-(3-(2,5-dioxo-2,5-dihydro-1H-pyrrol-1-yl)propanamido)-N-(2-(5-methoxy-1H-indol-3-yl)ethyl)-3,6,9,12-tetraoxapentadecan-15-amide as a yellow oil (210 mg, 92% yield). MS 589.2 (M+H⁺).

### Step 2. Preparation of (2R)-2-amino-3-((1-(1-(5-methoxy-1H-indol-3-yl)-4,20-dioxo-7,10,13,16-tetraoxa-3,19-diazadocosan-22-yl)-2,5-dioxopyrrolidin-3-yl)thio)propanoic acid

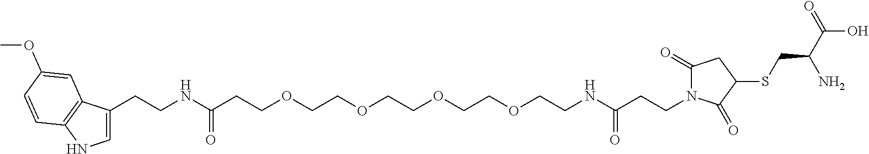 [SMILES: COc1ccc2[nH]cc(CCNC(=O)CCOCCOCCOCCOCCNC(=O)CCN3C(=O)CC(SC[C@H](N)C(=O)O)C3=O)c2c1]

1-(3-(2,5-dioxo-2,5-dihydro-1H-pyrrol-1-yl)propanamido)-N-(2-(5-methoxy-1H-indol-3-yl)ethyl)-3,6,9,12-tetraoxapentadecan-15-amide (229 mg, 0.389 mmol) was dissolved in N,N-dimethylformamide (dry) (5 mL) and combined with (R)-2-amino-3-mercaptopropanoic acid (189 mg, 1.556 mmol) and the mixture was stirred at rt overnight. The mixture was then concentrated in vacuo and purified by reversed phase flash chromatography (5-100% MeCN in H₂O) to afford (2R)-2-amino-3-((1-(1-(5-methoxy-1H-indol-3-yl)-4,20-dioxo-7,10,13,16-tetraoxa-3,19-diazadocosan-22-yl)-2,5-dioxopyrrolidin-3-yl)thio)propanoic acid (59 mg, 21.37% yield) as a white solid after lyophilization. MS 710.2 (M+H⁺).

### Example 3

### β-Amyloid Aggregation, Thioflavin T, Fluorescence Assay

In this Example, the activity of the melatonin-cysteine conjugates that were prepared in Examples 1 and 2 were tested to determine their ability to inhibit fibrillogenis to the activity of melatonin in the absence and presence of ApoE4.

In a NUNC PP 96-well plate 100 μL of MilliQ dH₂O was added to the outer wells surrounding the test wells in order to minimize evaporation. Samples in a final volume of 100 μl/well were added to the 96-well plate as follows:

TABLE 2
Thioflavin T Fluorescence Assay

| Sample No. | Contents |
|---|---|
| 1 | β-Amyloid 1-40 peptide |
| 2 | β-Amyloid 1-40 peptide and Melatonin |
| 3 | β-Amyloid 1-40 peptide; Melatonin; and ApoE4 |
| 4 | β-Amyloid 1-40 peptide and Example 1 |
| 5 | β-Amyloid 1-40 peptide; Example 1 and ApoE4 |
| 6 | β-Amyloid 1-40 and Example 2, |
| 7 | β-Amyloid 1-40; Example 2; and ApoE4 |

Ratios of components added to each well were as follows: 60 μM β-Amyloid 1-40: 60 μM Melatonin or Example 1 or Example 2: 0.727 μM ApoE4 or 12 μM β-Amyloid 1-40: 12 μM Melatonin or Example 1 or Example 2: 0.145 μM ApoE4. Aggregation was measured at 0 and 24 hours incubation time.

5 μL was obtained from each test sample after incubation and added to 2 mL of a glycine/NaOH buffer (50 mM, pH 9.2) containing 3 μM thioflavin T. Fluorescence intensities were measured at an excitation wavelength of 450 nm and an emission wavelength of 485 nm in a Envision fluorescence spectrophotometer. A time scan of fluorescence intensity was performed, and three measurements were taken after the decay reached a plateau at 200, 220, and 240 s and averaged after subtracting the background fluorescence of 3 μM thioflavin T in the blank buffers. Neither melatonin nor any of the other compounds used in this study exhibited significant fluorescence within the regions of interest at any time point. All measurements were taken in triplicate. Siliconized polypropylene microcentrifuge tubes (USA Scientific) were used for these experiments. Solutions of recombinant apolipoproteins were immediately lyophilized and, prior to being used, resuspended in 0.1 M tris-phosphate-HCl buffer (pH 7.4). Aqueous stock solutions of 1 mM melatonin were made by first preparing a 10 mM suspension of the hydrochloride salt of the hormone in 1 N HCl and then by completely dissolving it in 100 mM phosphate-buffered saline at pH 7.4 (1:10, v:v) and readjusting the pH to 7.4 with 1 N NaOH. Solutions of Aβ were prepared by dissolving 2.2 mg of the peptide in 1 mL of 50 mM buffer at pH 9.6. Aliquots (50 uL) of this solution were lyophilized and stored at −80° C. until they were needed for the experiments. Working stock solutions of the peptide (concentration of 500 uM) were prepared in HPLC-grade water immediately prior to the experiments. The absence of aggregates and amyloid fibrils in these solutions was verified by spectrofluorometry. In the experimental samples, Aβ was further diluted 1:1 with phosphate-buffered saline (pH 7.4, 100 mM) to which melatonin and/or apoE4 or equivalent volumes of buffer solution were added. The final concentration of Aβ in each sample was either 60 or 12 μM, and the melatonin:AB:apoE4 molar ratios were 100:100:0.83.

### Results

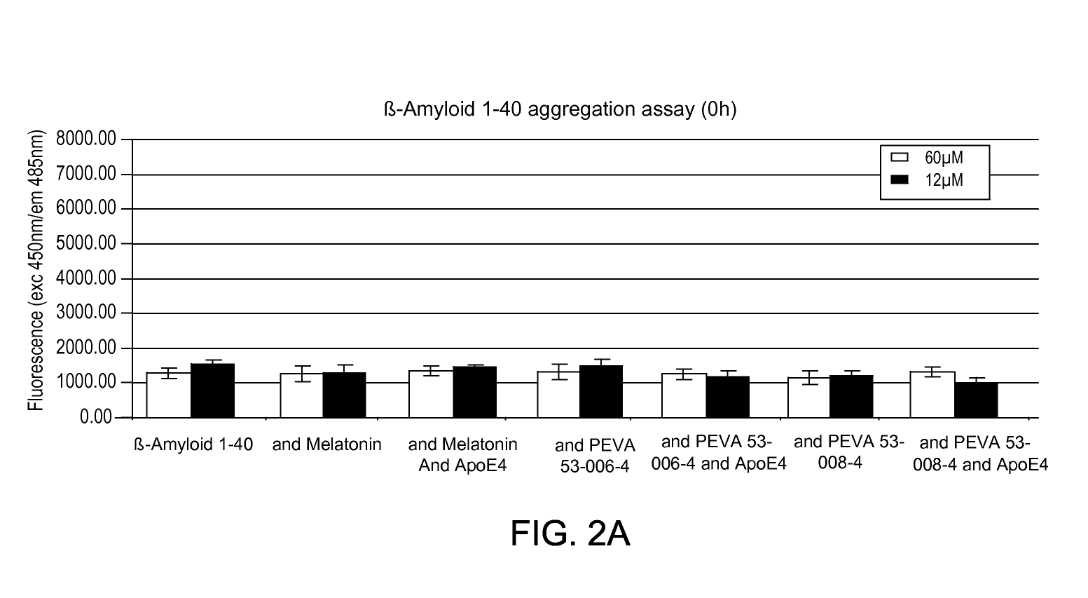

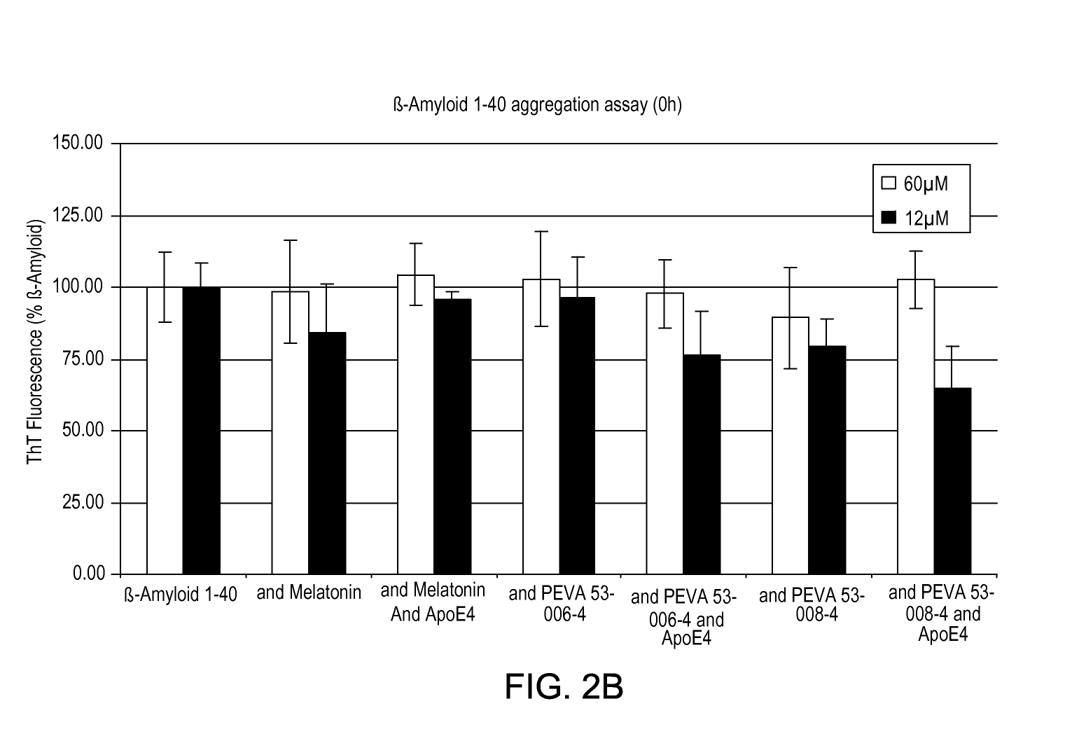

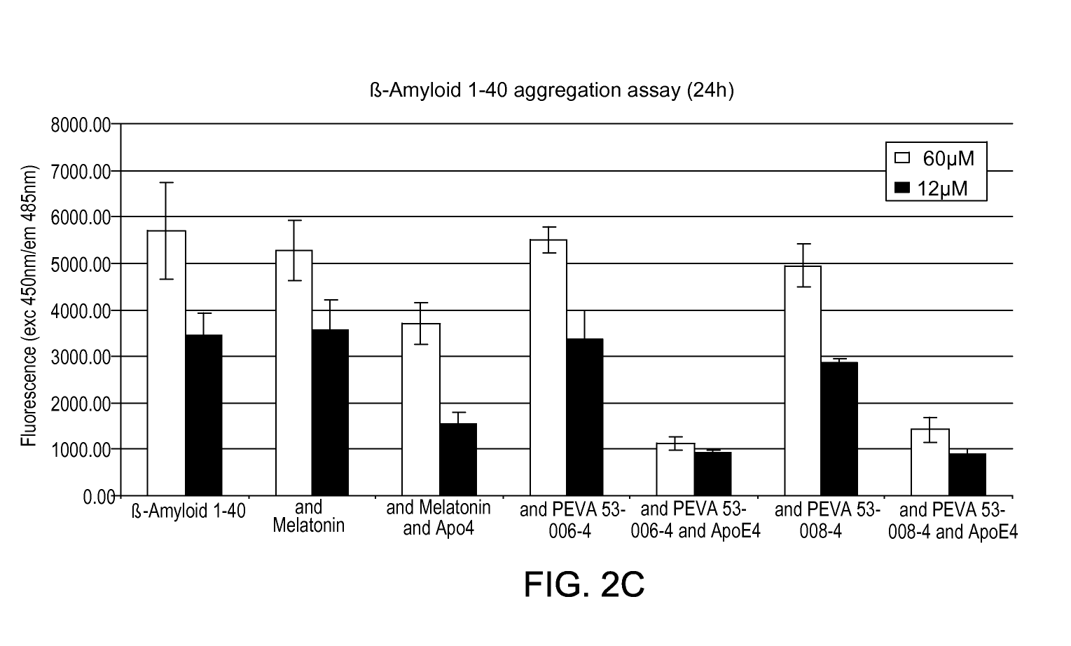

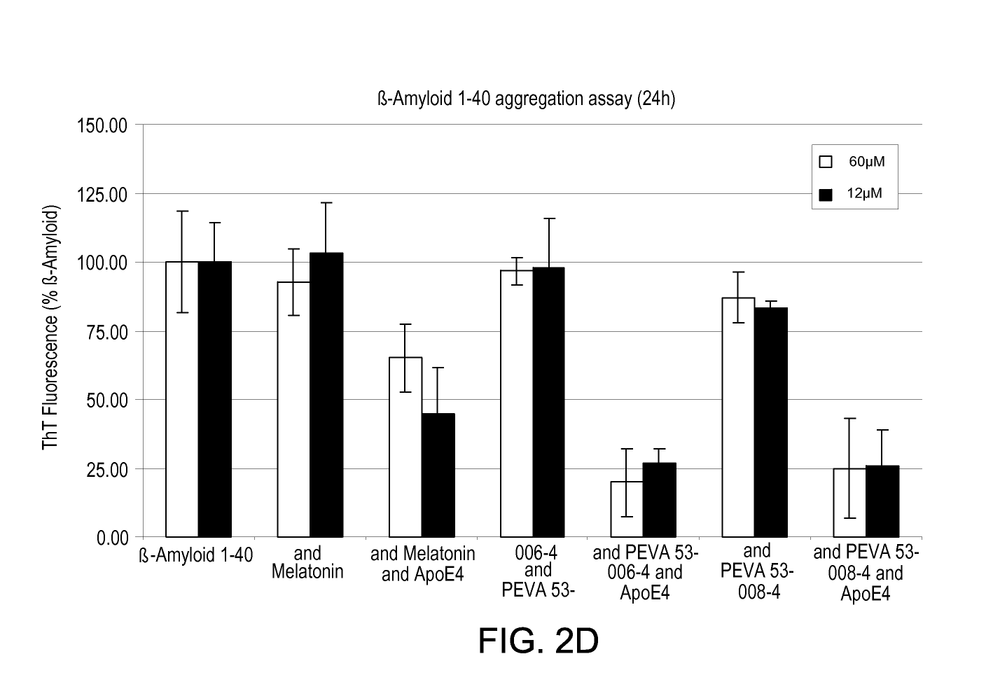

As shown in FIG. 2, none of the three compounds tested significantly reduced fibrillogenisis over a period of 24 hours when incubated at equimolar concentrations with Aβ alone. However, all the compounds reduced fibrillogenesis when ApoE4 was added to the mixtures. The amount of ApoE4 compared to the test compounds was about 1/80. Surprisingly, Example 1 and Example 2 were about 2 fold more potent than melatonin in reducing fibrillogenis in each case by 75%. These data suggest that melatonin-antibody conjugates made by the same methods will likely also exhibit this activity, and accordingly have therapeutic potential and may be advantageous compared to treatments that do not reverse the profibrilogenic properties of ApoE4.

### Example 4

### Oxygen Radical Absorbance Capacity Assay (ORAC Antioxidant Test)

### Materials:

Sodium Fluorescein was purchased from Invitrogen. 2,2′-Azobis(2-amidinopropane) dihydrochloride (AAPH), 6-hydroxy-2,5,7,8-tetramethylchroman-2-carboxylic acid (Trolox®), gallic acid, Epigallocatechin gallate (EGCG), epigallocatechin (EGC), and quercetin dihydrate were purchased from Sigma-Aldrich (St. Louis, Mo.). Black-sided, special optics clear bottom plates (part #3615) were obtained from Corning.

### Background:

The ORAC assay depends on the free radical damage to a fluorescent probe, such as fluorescein, to result in a downward change of fluorescent intensity. The assumption is that the degree of change is indicative of the amount of radical damage. The presence of antioxidants results in an inhibition in the free radical damage to the fluorescent compound. This inhibition is observed as a preservation of the fluorescent signal. Reactions containing antioxidants and or blanks are run in parallel using equivalent amounts of a molecule capable of generating super reactive oxygen species (ROS) and fluorescent probe. Because the reaction is driven to completion, one can quantitate the protection by calculating the area under the curve (AUC) from the experimental sample. After subtracting the AUC from the blank, the resultant difference would be the protection conferred by the antioxidant compound. Comparison to a set of known standards allows one to calculate equivalents and compare results from different samples and experiments. Typically Trolox®, (6-hydroxy-2,5,7,8-tetrametmethylchroman-2-carboxylic acid) a water soluble vitamin E analog, is used as the calibration standard and ORAC results are expressed as Trolox® equivalents. The ORAC assay is unique in that because the assay is driven to completion the AUC calculation combines both the inhibition time as well inhibition percentage of free radical damage by the antioxidant into a single quantity.

### Procedure:

Reactive oxygen species generators were added to parallel reactions containing equal amounts of a fluorescent probe. Reactions contained either a buffer blank or antioxidant samples and standards. The antioxidant capacity of a sample is the net difference between the area under the curve (AUC) of the sample and that of the blank. The exterior wells were not used for experimental determinations. 200 μL of MilliQ dH2O was added to the outer wells surrounding the test wells in order to minimize evaporation. 150 μL sodium fluorescein was added to experimental wells. In addition, blank wells received 25 μl of 75 mM phosphate buffer (BLANK), standards received 25 μL Trolox dilution (STD) and samples received 25 μL Melatonin in 75 mM phosphate buffer pH 7.4 (SAMPLES) in the Optiplate-96F solid black plates (Perkin Elmer). The plate was allowed to equilibrate by incubating for 30 min in the Victor2^{V} Microplate Reader (Perkin Elmer, Waltham, Mass.) at 37° C. Reactions were initiated by the addition 25 μL of AAPH solution (free radical initiator) for a final reaction volume of 200 μl. The fluorescence was monitored kinetically in Victor2V with temperature control reader for at least 80 min with data taken every minute. Fast orbital shaking (0.5 mm) for 10 sec was performed prior to each reading. The temperature was set to 37° C. at all time and the reagents were treated as light sensitive.

### Results:

The kinetic curves of several different concentrations of Trolox® standard demonstrates varying amounts of protection of fluorescein against oxidation that results in the loss of fluorescence. The highest concentration tested (100 μM) provided virtually full protection for approximately 20 minutes, before fluorescence intensity began to diminish, while the lowest concentration tested (6.25 μM) provided only slight protection above the buffer only control.

As shown in Tables 3-5, the melatonin conjugates prepared in Examples 1-2 were shown to exhibit better antioxidant activity than Melatonin.

TABLE 3
ORAC assay measurements for Melatonin

| | Trolox | | Melatonin | |
|---|---|---|---|---|
| Concentration | AUC | Net AUC | AUC | Net AUC |
| 0 | 6.65 | 0.00 | 6.65 | 0.00 |
| 0.390625 | 11.95 | 5.30 | 14.74 | 8.09 |
| 0.78125 | 14.90 | 8.24 | 20.89 | 14.24 |
| 1.5625 | 20.00 | 13.34 | 28.74 | 22.09 |
| 3.125 | 28.96 | 22.31 | 42.59 | 35.94 |
| 6.25 | 44.91 | 38.25 | 66.10 | 59.45 |
| 12.5 | 77.61 | 70.96 | 88.65 | 82.00 |
| 25 | 96.34 | 89.68 | 93.12 | 86.47 |
| 50 | 98.05 | 91.40 | 94.75 | 88.10 |
| 100 | 95.82 | 89.17 | 91.48 | 84.82 |

Calculated Trolox® equivalents for Melatonin: 1.65. Trolox® equivalents are calculated as follows:

AUC=(R1/R1)+(R2/R1)+(R3/R1)+ . . . +(Rn/R1) (Eq. 1)

Where R1 is the fluorescence reading at the initiation of the reaction and Rn is the last measurement.

Net AUC=AUC(sample)−AUC(blank) (Eq. 2)

A standard curve was obtained by plotting the Net AUC of different Trolox® concentrations against their concentration resulting in linear relationship. The resultant standard curve was then interpolated to determine of antioxidant capacity of the samples and reported as Trolox® equivalence. To determine Trolox® equivalents of each sample range the ratio of the slope (m) of the linear regression analysis of the sample to the slope of the linear regression of Trolox® was used:

TE(range of concentrations)=m(sample)/m(Trolox®) (Eq. 3)

TABLE 4
ORAC assay measurements for Example 1 conjugate

| | Trolox | | Example 1 conjugate | |
|---|---|---|---|---|
| Concentration | AUC | Net AUC | AUC | Net AUC |
| 0 | 6.43 | 0.00 | 6.43 | 0.00 |
| 0.390625 | 13.53 | 7.10 | 14.99 | 8.56 |
| 0.78125 | 15.01 | 8.58 | 20.01 | 13.58 |
| 1.5625 | 19.69 | 13.26 | 29.51 | 23.08 |
| 3.125 | 29.49 | 23.06 | 46.55 | 40.12 |
| 6.25 | 44.55 | 38.12 | 74.68 | 68.25 |
| 12.5 | 74.88 | 68.45 | 93.56 | 87.13 |
| 25 | 100.28 | 93.85 | 93.03 | 86.60 |
| 50 | 97.48 | 91.05 | 88.39 | 81.96 |
| 100 | 99.05 | 92.62 | 82.12 | 75.69 |

Calculated Trolox® equivalents for Example 1 conjugate: 2.02.

TABLE 5
ORAC assay measurements for Example 2 conjugate

| | Trolox | | Example 2 conjugate | |
|---|---|---|---|---|
| Concentration | AUC | Net AUC | AUC | Net AUC |
| 0 | 6.23 | 0.00 | 6.23 | 0.00 |
| 0.390625 | 11.49 | 5.27 | 13.97 | 7.75 |
| 0.78125 | 14.62 | 8.39 | 20.49 | 14.27 |
| 1.5625 | 19.52 | 13.30 | 28.06 | 21.84 |
| 3.125 | 28.51 | 22.29 | 45.62 | 39.39 |
| 6.25 | 43.41 | 37.18 | 71.79 | 65.56 |
| 12.5 | 73.04 | 66.81 | 94.48 | 88.26 |
| 25 | 98.61 | 92.39 | 97.13 | 90.91 |
| 50 | 100.67 | 94.45 | 97.12 | 90.90 |
| 100 | 97.58 | 91.35 | 96.55 | 90.33 |

Calculated Trolox® equivalents for Example 2 conjugate: 1.97.

### Prophetic Example 1

### Preparation of N01-OX2 Antibodies

Melatonin (Sigma) is conjugated to a chimeric antibody targeted to the N-terminus of Aβ peptide (N01) (Ab) using an SMCC linker. SMCC is dissolved in dimethylacetamide (DMA) and added to the antibody solution to make a final SMCC/Ab molar ratio of 10:1. The reaction is allowed to proceed for 3 hours at room temperature with mixing. The SMCC-modified antibody is subsequently purified on a GE Healthcare HiTrap desalting column (G-25) equilibrated in 35 mM sodium citrate with 150 mM NaCl and 2 mM EDTA, pH 6.0. The drug (e.g. melatonin) is dissolved in DMA, and added to the SMCC-antibody preparation to give a molar ratio of melatonin to antibody of 10:1. The reaction is allowed to proceed for 4 to 20 hours at room temperature with mixing. The melatonin-modified antibody solution is diafiltered with 20 volumes of phosphate-buffered saline to remove unreacted melatonin, sterile-filtered, and stored at 4° C. Typically, a 40% to 60% yield of antibody is achieved through this process. The preparation is usually greater than 95% monomeric as may be assessed by gel filtration and laser light scattering. Typically, the drug to antibody ratio is expected to be between about 2.5 and 4.5. These same conditions can also be used to conjugate the constructs prepared in Examples 1-2 with a chimeric antibody targeted to the N-terminus of Aβ peptide (N01) (Ab).

### Prophetic Example 2

### Confirmation of Reactivity of N01-OX2 Antibodies

The ADC prepared in Prophetic Example 1 is assayed to confirm reactivity with the intended target epitope (N-terminus peptide of Aβ peptide). The ADC are subjected to Western blotting against each peptide of amyloid β (1-40), amyloid β (2-40), and amyloid β (3-40) according to known methods for carrying out Western blotting. For comparison, Chinese hamster cells in which amyloid β precursor proteins (APP) are forcibly expressed are homogenized with a buffer solution containing 1% Triton and centrifuged to obtain a supernatant liquid, which may be subjected to Western blotting in the same manner as above. This supernatant is expected to contain, in addition to APP, amyloid β and pc terminal fragments (βCTF) cut from APP at the β site. In addition, reactivity of each ADC can be compared to that of unconjugated N01.

Binding affinity of the ADC prepared in prophetic Example 1 may be assayed using binding inhibition assays known in the art. See, e.g., Johnson-Wood, K. et al. (1997) Proc. Natl. Acad. Sci. USA 94:1550-1555.

### Prophetic Example 3

### In Vivo Assay for N01-OX2

N01-OX2 ADC obtained in Prophetic Example 1, unconjugated N01 antibody, or melatonin alone is diluted with PBS to a concentration of 1 mg/ml and abdominally injected into 16-month old Tg2576 genetically engineered mice exhibiting amyloid precursor proteins (APP), at a dose of 10 mg/Kg (body weight) once per week. As a control, some mice receive only PBS administered in the same manner. After 12 injections, mice are sacrificed and the left cerebral hemisphere is fixed with a 4% formaldehyde buffer solution and paraffin was embedded. 5 micron continuous segments are prepared from the paraffin-embedded cerebral tissue and immunologically stained with amyloid β (1-40) polyclonal antibody and amyloid β (1-42) polyclonal antibody (both manufactured by Immuno-Biological Laboratories Co., Ltd., product numbers 18580 and 18582).

Segments from the cortical layer and from the hippocampus are subjected to imaging analysis using a microscopic digital camera and simple PCI software (Compix, Inc. Imaging Systems, USA). Deposition of amyloid β (1-40) and amyloid β (1-42) is determined as the proportion (%) of the immunological staining positive region in the entire region.

In addition, insoluble amyloid β in a 0.05 M Tris HCl buffered physiological saline solution (TBS, pH 7.6) is extracted from the frontal right of the head (¼ of the brain) with 6 M guanidine-hydrochloric acid to measure amyloid β (1-42) using an assay kit of amyloid β (1-42) and human amyloid β (1-42) (manufactured by Immuno-Biological Laboratories Co., Ltd., Product No. 17711) in a sandwich ELISA system. The method described by M. Morishima, Kawashima, et al., in Am. J. Pathol., 157 (2000) 2093-2099 can be followed for extraction.

It is expected that the number of senile plaques is smallest in the brain of mice to which the N01-OX2 ADC was administered as compared to the other treatment groups. The number of senile plaques in the basal nuclei, in which the amyloid β deposits is slower than in the cortical layer or hippocampus, is also expected to be smallest in N01-OX2 ADC treated mice. It is also expected that the amount of insoluble amyloid β extracted and measured by ELISA, as described above, will be smallest in the IN-N01-OX2 treated group.

### Prophetic Example 4

### In Vivo Assay of N01-OX2 in Blue Light AMD Model

In this Example, the ADC prepared in Prophetic Example 1 is tested for its ability to protect retinal epithelial cells from oxidative damage. This example evaluates the prevention of photoreceptor damage following exposure to intense blue light. This model creates significant photo-oxidative stress and approximates the pathology associated with geographic atrophy, the late stage of Dry Age Related Macular Degeneration (AMD).

42 Sprague Dawley rats are acclimated to institution lighting for a minimum of 21 days prior to light exposure. Light exposure is conducted over 2 sessions with half of each study group challenged during each session (a total of 18 animals are exposed to blue light damage in each session). Prior to light exposure animals are dark adapted overnight. Ocular assessments begin after a minimum 5 day recovery period following light exposure. The details of this study are illustrated in Table 6 below.

TABLE 6
Experimental Parameters of N01-OX2 in Blue Light AMD model

| Group | N | Test |
|---|---|---|
| 2 | 10 | Test Article 1:5 μl intravitreal injection both eyes on Day -2 |
| 3 | 10 | Test Article 1:5 μl intravitreal injection both eyes on Day -2 |
| 4 | 8 | Vehicle: 1:5 μl intravitreal injection both eyes on Day -2 |
| 5 | 8 | 8-OH-DPAT 5 mg/kg. Once daily IP on Days -2 through Day +2 |
| 6 | 3-6 | Untreated, unexposed Controls |

Optical Coherence Tomography (OCT) imaging is conducted on all animals at Day >7 following a recovery period from light exposure. OCT and fundus images are collected using the Spectralis HRA+OCT (Heidelberg Engineering). Corneas are anesthetized by topical administration of 1-2 drops of Proparacaine and pupil dilation is achieved with 1-3 drops each of 1% Tropicamide and 2.5% Phenylephrine. The animals are anesthetized with isoflurane to effect. Four OCT slices are collected from each eye, approximately 15° from the Optic Nerve in the superior nasal, superior temporal, inferior nasal, and inferior temporal quadrants. Thickness measurements are made from each quadrant.

Electroretinography (ERGs) is performed on all animals at Day >8 following OCT measurements. The ERG exam is conducted using the Espion E2 ERG recording system (Diagnosys, LLC). Corneas are anesthetized by topical administration of 1-2 drops of Proparacaine and pupil dilation is achieved with 1-3 drops each of 1% Tropicamide and 2.5% Phenylephrine. Dark adapted ERG's are recorded according to established protocols.

Any chemical modification of the payload requires careful evaluation to determine the effect of the modification. In addition, it is necessary to evaluate the effect of linking the compound to cysteine or lysine residues depending on which amino acid is used to conjugate the compound to antibody. Thirdly, it is necessary to evaluate the neuroprotective properties after conjugation to the antibody. Finally, it is necessary to demonstrate that the compound and method of conjugation do not significantly affect the affinity of the antibody or cause it to aggregate.

Depending on the mechanism of action of the payload, one would evaluate its neuroprotective functions using one or more assays described in Table 7.

TABLE 7
Evaluation of Payload

| Properties Measured | Assay |
|---|---|
| Free radical scavenging activity | Free radical scavenging activity assays: |
| | e.g. Oxygen radical absorbance capacity assay |
| Reduced oxidative stress in vivo | Evaluate markers of oxidative stress in brain |
| | of transgenic models (AD, HD & PD) by ELISA |
| | assays and immunohistochemistry |
| | a. iPF_{2α}-VI (a reliable biomarker of lipid |
| | peroxidation) |
| | b. protein carbonyls (known biomarkers of protein |
| | oxidation) |
| Reduced inflammation in vivo | Evaluate markers of inflammation in brain of transgenic |
| | models (AD, HD & PD) by ELISA |
| | assays, Reverse transcription PCR and |
| | immunohistochemistry |
| | a. IL-1β |
| | b. IL-6 |
| | c. TNFα |
| | d. YMI |
| | e. AGl |
| | f. GFAP |
| | g. Mrcl and CD163 known to be restricted to cerebral |
| | vasculature |
| Inhibition of fibrillogenesis | Thioflavin assay |
| Reduced amyloid production | Western blot analysis in supernatants from neurons in |
| | culture |
| Reduced amyloid deposits in vivo | Quantitative immunohistochemistry in fixed tissue |
| | sections of transgenic mice or ELISA of brain |
| | homogenate to measure effect on amyloid deposition |
| Promotion of neurogenesis | ELISA based format to measure effects on neural |
| | differentiation potential quantified by the use of three |
| | validated lineage specific monoclonal antibodies, |
| | anti-bIII-tubulin for neurons, anti-glial fibrillary acidic |
| | protein (GFAP) for astrocytes and anti-CNPase for |
| | early oligodendrocytes, in an ELISA based format. |
| | Also included are control mouse IgG and an |
| | anti-GAPDH monoclonal antibody to correct for |
| | background signals along with a cell stain solution to |
| | help normalize for variations in the cell numbers across |
| | samples. Cells are cultured and fixed in a standard 96 |
| | well plate with the lineage analysis done directly on the |
| | whole cell population. Immunocytochemistry or |
| Blocked hyperphosphorylation of tau | ELISA with phospo tau antibodies |
| Improved cognitive function | Behavioral assays in transgenic models (e.g. Morris- |
| | Water Maze, Fear Conditioning, Y-maze, Radial Arm Maze |
| Increased glucose utilization in the brain | 18F-FDG μPET metabolic imaging of glucose uptake or |
| | autoradiography of fixed tissue sections from |
| | transgenic models |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An antibody-drug conjugate comprising an antibody targeted to an amyloidogenic polypeptide or a tau polypeptide conjugated to a cytoprotective agent.

2. The antibody-drug conjugate according to claim 1, wherein the cytoprotective agent is melatonin or a derivative thereof.

3. The antibody-drug conjugate according to claim 2, wherein the melatonin is conjugated to an antibody targeted to an amyloid beta polypeptide.

4. The antibody-drug conjugate according to claim 1, wherein said amyloidogenic polypeptide is an amyloid beta peptide, or a biologically active fragment thereof.

5. The antibody-drug conjugate according to claim 1, wherein said amyloidogenic polypeptide is an amyloid-associated polypeptide selected from the group consisting of protease inhibitor alpha 1-antichymotrypsin, apolipoprotein E (apoE), and EpoE4.

6. The antibody-drug conjugate according to claim 1, wherein said tau polypeptide is hyperphosphorylated.

7. The antibody-drug conjugate according to claim 1, wherein said amyloidogenic polypeptide is selected from the group consisting of prion (PrPSc), amylin, calcitonin, atrial natriuretic factor (AANF), apolipoprotein AI, serum amyloid A, medin, transthyretin, lysozyme, beta 2 microglobulin (Aβ2M), gelsolin, keratoepithelin, cystatin, immunoglobulin light chain AL, α-synuclein, Huntingtin, and superoxide dismutase.

8. The antibody-drug conjugate according to claim 1, wherein said antibody is humanized.

9. The antibody-drug conjugate according to claim 1, wherein the antibody is a monoclonal antibody, a humanized antibody, a chimeric antibody, a bispecific antibody, an artificial antibody, a scFv antibody or a F(ab), or fragment thereof.

10. The antibody-drug conjugate according to claim 1, wherein said antibody is a camel antibody.

11. The antibody-drug conjugate according to claim 1, wherein said cytoprotective agent is an antioxidant.

12. The antibody-drug conjugate according to claim 11, wherein said antioxidant is selected from the group consisting of melatonin, indole-3-propionic acid, an indole amine, an indole acid, vitamin E, vitamin C, lipoic acid, uric acid, curcumin, glutathione, a polyphenol, a flavonoid, an anthraquinone methylthioninium chloride, dimebone, idebenone, a rhodamine, an insulin sensitizer, an 8-hydroxyquinolone derivative, PBT2, PBT434, penicillamine, Trientine, a tetracycline, (N-(pyridin-2-ylmethyl)aniline), N1,N1-dimethyl-N4-(pyridin-2-ylmethyl)benzene-1,4-diamine, 2,6-diaminopyridine, AZD-103, cyclohexane-1,2,3,4,5,6-hexyl, myo-inositol, scyllo-inositol, methylene blue, TRx0014 and an NO scavenger.

13. The antibody-drug conjugate according to claim 1, wherein said antibody is conjugated to said cytoprotective agent by a linker.

14. The antibody-drug conjugate according to claim 13, wherein said linker is selected from the group consisting of a maleimide linker, hydrazone linker, disulfide linker, thioether linker, and peptide linker.

15. The antibody-drug conjugate according to claim 13, wherein said linker is cleavable under intracellular conditions.

16. The antibody-drug conjugate according to claim 15, wherein the cleavable linker is a peptide linker cleavable by an intracellular protease.

17. The antibody-drug conjugate according to claim 14, wherein the peptide linker is a dipeptide linker.

18. The antibody-drug conjugate according to claim 17, wherein said dipeptide linker is a citrulline-valine based linker.

19. The antibody-drug conjugate according to claim 1, further comprising a marker.

20. The antibody-drug conjugate according to claim 19, wherein the marker is selected from the group consisting of an isotope, a radiolabel, a fluorescent label, and an enzyme that catalyzes a detectable modification to a substrate.

21. The antibody-drug conjugate according to claim 20, wherein said marker is conjugated to said cytoprotective agent.

22. The antibody-drug conjugate according to claim 20, wherein said marker is incorporated into the cytoprotective agent during synthesis of the cytoprotective agent.

23. A pharmaceutical composition comprising an antibody-drug conjugate according to any one of the preceding claims and a pharmaceutically acceptable carrier.

24. A method for detecting an amyloid deposit in a subject, comprising administering the antibody-drug conjugate according to claim 19 to the subject, and detecting the presence of the marker, wherein the subject has an amyloid deposit if the marker is detected in the subject.

25. A method for inhibiting accumulation of an amyloidogenic polypeptide in the brain of a patient suffering from a proteinopathy, comprising administering to said patient a therapeutically effective amount of the antibody-drug conjugate according to claim 1.

26. The method according to claim 25, wherein the composition is administered intravenously.

27. The method of claim 26, wherein said proteinopathy is selected from the group consisting of age related macular degeneration (AMD), glaucoma, traumatic brain injury, cerebral amyloid angiopathy (CAA), hereditary cerebral hemorrhage with amyloidosis Dutch type (HCHWA-D) Alzheimer's disease, early onset familial Alzheimer's disease (EOFAD), Down Syndrome, Parkinson's disease (PD), Huntington's disease (HD), amyotrophic lateral sclerosis (ALS), transmissible spongiform encephalopathy, Pick's complex, and prion disease.

28. A method for promoting clearance of aggregates from the brain of a subject, comprising administering to the subject the antibody-drug conjugate according to claim 1, wherein said polypeptide is tau, under conditions and in an amount effective to promote clearance of neurofibrillary tangles from the brain of the subject.

29. A method for treating or delaying onset of a proteinopathy, comprising administering to a subject in need thereof an effective amount of the antibody-drug conjugate according to claim 1 for inhibiting the formation of fibrils, the formation of amyloid or amyloid-like deposits, or to inhibit the formation of neurofibrillary tangles.

30. The method of claim 29, wherein said proteinopathy is selected from the group consisting of age related macular degeneration (AMD), glaucoma, traumatic brain injury, cerebral amyloid angiopathy (CAA), hereditary cerebral hemorrhage with amyloidosis Dutch type (HCHWA-D) Alzheimer's disease, early onset familial Alzheimer's disease (EOFAD), Down Syndrome, Parkinson's disease (PD), Huntington's disease (HD), amyotrophic lateral sclerosis (ALS), prefrontal dementia, Pick's complex, and prion disease.

31. The method of claim 24, wherein the subject is a mammal.

32. The method of claim 24, wherein the mammal is a human.

33. The method of claim 24, wherein the composition is administered subcutaneously, intravenously, intradermally, intramuscularly, intraperitoneally, intracerebrally, intranasally, orally, transdermally, buccally, intra-arterially, intracranially, or intracephalically.

34. The antibody-drug conjugate according to claim 1, wherein the antibody is conjugated to a construct comprising the structure:

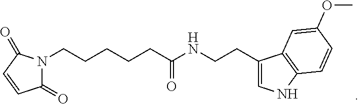 [SMILES: COc1ccc2[nH]cc(CCNC(=O)CCCCCN3C(=O)C=CC3=O)c2c1]

35. The antibody-drug conjugate according to claim 1, wherein the antibody is conjugated to a construct comprising the structure:

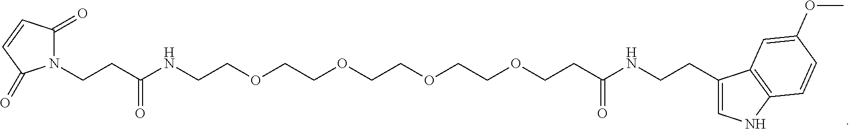 [SMILES: COc1ccc2[nH]cc(CCNC(=O)CCOCCOCCOCCOCCNC(=O)CCN3C(=O)C=CC3=O)c2c1]

36. The antibody-drug conjugate according to claim 12, wherein the insulin sensitizer is pioglitazone or rosiglitazone.


SEQUENCE LISTING (13 sequences)

SEQ ID NO: 1 - protein, 4 aa, Artificial Sequence
  Description of Artificial Sequence Synthetic peptide
ALAL

SEQ ID NO: 2 - protein, 4 aa, Artificial Sequence
  Description of Artificial Sequence Synthetic peptide
ALAL

SEQ ID NO: 3 - protein, 6 aa, Artificial Sequence
  Description of Artificial Sequence Synthetic peptide
PVGLIG

SEQ ID NO: 4 - protein, 5 aa, Artificial Sequence
  Description of Artificial Sequence Synthetic peptide
GPLGV

SEQ ID NO: 5 - protein, 8 aa, Artificial Sequence
  Description of Artificial Sequence Synthetic peptide
GPLGIAGQ

SEQ ID NO: 6 - protein, 4 aa, Artificial Sequence
  Description of Artificial Sequence Synthetic peptide
PLGL

SEQ ID NO: 7 - protein, 8 aa, Artificial Sequence
  Description of Artificial Sequence Synthetic peptide
GPLGMLSQ

SEQ ID NO: 8 - protein, 8 aa, Artificial Sequence
  Description of Artificial Sequence Synthetic peptide
GPLGLWAQ

SEQ ID NO: 9 - protein, 4 aa, Artificial Sequence
  Description of Artificial Sequence Synthetic peptide
ALAL

SEQ ID NO: 10 - protein, 4 aa, Artificial Sequence
  Description of Artificial Sequence Synthetic peptide
GFLG

SEQ ID NO: 11 - protein, 4 aa, Artificial Sequence
  Description of Artificial Sequence Synthetic peptide
LLGL

SEQ ID NO: 12 - protein, 3 aa, Artificial Sequence
  Description of Artificial Sequence Synthetic peptide
KLV

SEQ ID NO: 13 - protein, 4 aa, Artificial Sequence
  Description of Artificial Sequence Synthetic peptide
ALAL